(19) United States
(12) Patent Application Publication
(10) Pub. No.: US 2011/0184465 A1
(43) Pub. Date: Jul. 28, 2011

(54) UNIQUE MINIMALLY INVASIVE SYSTEM FOR SECURING CONNECTING ROD ELEMENT TO TRAILING ENDS OF ADJACENT PEDICLE SCREWS

(76) Inventor: Frank H. Boehm, Utica, NY (US)

(21) Appl. No.: 13/013,504

(22) Filed: Jan. 25, 2011

Related U.S. Application Data
(60) Provisional application No. 61/282,334, filed on Jan. 25, 2010.

(51) Int. Cl. A61B 17/70; A61B 17/56; A61B 17/86

(52) U.S. Cl. 606/264; 606/86 A; 606/329

(57) ABSTRACT

A spinal stabilizing system with at least one connecting rod-like elongated member; a plurality of bone fastening screws that are implantable into the pedicles of the superior and inferior vertebrae comprising at least one target motion segment, wherein said screws being then coupleable to said connecting rod and this system thereby being capable of stabilizing and hence eliminating any between the vertebrae of the target motion segment and wherein the trailing ends of said bone fastening screws is provided with a cradle which couples with said connecting rod-like elongated member.

### FIELD OF INVENTION

The invention relates to the general field of spinal surgery and specifically to a system of devices and methods for use which provides a surgeon with the ability to establish a pedicle-screw based stabilization of one or more target motion segments utilizing a surgical technique that accomplishes the stated surgical goal with minimal perturbation of the associated soft tissues.

### HISTORY AND RELATED ART

The human spine is a complex, superbly (perhaps divinely) engineered structure of 24 mobile vertebrae forming an articulated column and divided into 3 general anatomic segments: the cervical spine composed of 7 vertebrae, the 12 thoracic vertebrae, and the 5 lumbar vertebrae. From the perspective of nomenclature, the segments are referred to by the letters C, T, or L corresponding to the segment in question, as well as a number reflecting the position of the vertebra[e] of interest as ordered from cranial to caudal position within that segment. The most caudal aspect of this column is a single structure representing 9 fused vertebrae, the sacro-coccygeal segments.

With the exception of the relationship between the first and second vertebrae at the cranial most aspect of the spine (C1-2), the articulation, or junction, of each pair of the remaining mobile vertebrae is represented anteriorly by a disc joint, or intervertebral disc, and posteriorly by the paired facet joints. Such an arrangement also is found at the junction of the lumbar and sacral segments. These joints govern the motion necessary at each level in order to provide the entire column with the flexibility (and hence the strength) to face the challenges of existence as a vertebrate.

Mankind, as a species, is unique in the vertebrate kingdom insofar we stand upright and ambulate in a truly bipedal fashion; this likely contributes to the fact that the spine is affected by numerous disease processes and, in fact, is the most common site of structural/anatomic causes of human suffering. Worldwide, degeneration of the intervertebral disc appears to be responsible for the [vast] majority of such suffering, although other issues, including facet degeneration, arthritic deposits on the spine causing narrowing (stenosis) of the canals provided for the spinal cord and/or the nerves also contribute significantly to the pathologic states of the spine. In order to understand spinal pathology better, a paradigm known as the “Motion Segment” is convenient and commonly used. This term refers to any pair of adjacent, articulating vertebrae with their associated intervertebral disc, facet joints, muscles tendons, ligaments, and nerves.

With the advent of the use of diagnostic X-rays in the early 20^{th} century, a scientific explanation of low back pain could (after 6 millennia of recorded history and medical “observations” and “cures,”) at last be attempted. Early X-rays allowed physicians to document the evolution of radiographic changes associated with back pain. Keen observers recognized that in some patients who demonstrated auto fusion reported resolution of their pain. Fusion, for the purposes of definition, refers to a process by which one or more vertebrae (or any plurality of bones, for that matter) biologically unite into functionality a single bone or unit. Auto fusion refers to a naturally-occurring process, while surgical fusion refers to a procedure by which a surgeon creates a milieu in which one or more separate bones unite into a single unit.

The observation that auto fusion resulted in relief of back pain in some patients ultimately led to the theoretical basis for spinal fusion (which had already been introduced by Hibbs in 1911 for tuberculosis) for degenerative disease. Since that time, spinal fusion has been one of the most common elective surgical procedures for many decades.

Part of the procedure that surgeons undertake to create a fusion is to place some form of bony substrate known as graft material in between the bones which are being fused. One of the challenges in spinal fusion is actuall_{y} accomplishing the maturation of the graft material placed at the time of surgery into a living, metabolizing, and structurally competent bridge of bone between the target vertebrae. For years, fusion rates of 60-65% were considered acceptable if not outstanding.

The technique of placing screws into the posterior aspect of the vertebra dates as far back as 1944, with the use of posterior vertebral screws as described by King. The actual technique of passing screws into the pedicles (the anatomic component of the vertebra that connects the posterior portion of the vertebrae to the expanded anterior vertebral bodies) and stabilizing the motion segment by then connecting the screws to a plate or rod was first described by Boucher in 1959, and later by Pennel in 1964. The great surgical pioneer Roy-Camille championed this technique as far back as 1963, and he published his first findings in 1970.

The great American Orthopedic Spine surgeon Leon Wiltse also promoted the concept of pedicle screw and plate/rod fixation. It appears that both Roy-Camille and Wiltse were considerably advanced in their thinking and, to utilize a popular euphemism, “ahead of their time.” Other approaches have included: Goldthwait, J. E. (1911). The Lumbosacral Articulation. An explanation of many cases of “lumbago,” “sciatica” and paraplegia. Boston Medical Journal, Vol. 64, p. 365-372. [0004] Ghormley, R. K. (1933). Low back pain with special reference to the articular facets, with presentation of an operative procedure. Journal of the American Medical Association. Vol. 101, p. 1773-1777. [0005] Putti, V. (1927). New Conceptions in the Pathogenesis of Sciatic Pain. Lancet. Vol. 2, p. 53-60. [0006] Williams, P. C. and Yglesias L. (1933). Lumbosacral Facetectomy for Post-fusion Persistent Sciatica. Journal of Bone and Joint Surgery. Vol. 15, p. 579. [0007] Shealey, C. N. (1976). Facet denervation in the management of back and sciatic pain. Clinical Orthopedics, 115, p. 157-164. [0008] Bogduk, N. (1983). The innervation of the lumbar spine. Spine, 8, 286-293.

These surgeons proposed a system of spinal stabilization (ultimately being disclosed herein) known as a so-called “Pedicle-Screw Based System,” insofar that it involves the stabilization or elimination of any motion occurring between at least two adjacent, articulating vertebrae [target vertebrae]. This goal is accomplished by implanting bone fastening screws into the pedicles [bilaterally] of said vertebrae, and then, in turn, securing the trailing ends of said bone fastening screws on each side to an immobile elongated element or connecting rod element which spans the distance between said screws. Such a system therefore eliminates the movement of [at least two] target vertebrae with respect to each other, as well as any movement of the intervening intervertebral disc and other associated soft tissues collectively referred to as the “Motion Segment” [and in the case of surgical intervention the “target” motion segment]; thus it is said to stabilize the target motion segment[s].

For the purpose of clarification, it is to be noted that when the screws and rods have been completely assembled in their entirety for the purpose of promoting a fusion within a patient, that assembly is referred to by the term construct.

However, this technique was popularized and truly came into its own by the designs, as well as the persona, of the immortal spine surgeon Dr. Arthur Steffee, and placement of pedicle screws to promote better fusion rates has now become the “platinum” standard of care in spinal fusion technology. In prior art, including U.S. Pat. No. 4,655,199, 4,696,290, 4,719,905, and 4,854,311 as well as others, Steffee teaches that when attempting to fuse a segment of the spine, stabilization of the target segment with a pedicle screw system results in improved fusion rates. Multiple studies have been published to demonstrate this to be true, with fusion rates rising from 55% to 80% or better.

As originally proposed, the surgical procedure of creating a fusion of at least one target motion segment was a major surgical procedure. This involved an operative field that provided visualization of the posterior aspects of the vertebrae to be fused, including the laminae, facets, and transverse processes. This, in turn, required removing or “stripping” the muscular attachments to all of these structures, as well as removing the muscles and soft tissues that lie between the transverse processes. The posterior cortical surfaces of these bony structures were then removed by bone-biting instruments or drills, pedicle screws were placed, and graft material was then placed in a fashion so as to grow between the transverse processes, the facets, and the laminae (if the laminae had not been removed for decompressive purposes). The pedicle screws were then placed, and the operation was completed. The placement of pedicle screws involved identifying certain landmarks as described originally by the aforementioned surgeons. The most common landmark was the junction of the transverse process and the facet joint and directing the screw somewhat medially as it was passed into the bone; passing the screw directly through the facet joint and directing it in a less medial and more anterior fashion was also described.

Ideally, the pedicle screw is introduced into the most posterior aspect of the pedicle, found at the junction of the transverse process and the facet joint. The screw is then advanced through the pedicle, and ultimately placement should result in the leading end of the screw being positioned within the vertebral body with the shaft being contained entirely within the pedicle.

The present invention incorporates by reference an application titled “Apparatus and method for treating the spine” filed with the United States Patent and Trademark Office Dec. 20, 2004, and published Aug. 4, 2005, with publication # 20050171549, the contents of which are contained herein by reference

The great concern in placing such screws is the possibility of creating an injury to the delicate neurologic structures such as the nerve roots and cauda equina, which intimately relate to the pedicles. Inadvertent placement of the screw in such a fashion that the screw did not remain completely with the pedicle but rather violated the wall of the pedicle easily resulted in injury to such structures.

As these techniques evolved further, such a fusion was often supplemented by removing at least part of the intervertebral disc at the target level, and placing bone in the intervertebral disc so as to fuse the vertebral bodies, in addition to the posterior bony structures. This technique became known as an “Interbody Fusion.” Performing an interbody fusion from a posterior approach is known as a “Posterior Lumbar Interbody Fusion” and often referred to by the abbreviated euphemism “PLIF.”

Still later, anterior approaches (first proposed for the lumbar spine by Cloward in the 1950's but later abandoned) were re-introduced into the armamentarium of the spinal surgeon. Such approaches became known as “Anterior Lumbar Interbody Fusion” or “ALIF.”

In the early part of the 21^{st} century, many factors have come together to attempt to drive surgical techniques in all areas of surgical practice towards “minimally-invasive” methods of accomplishing the goals of the surgery. One of the challenges of spinal surgery as this Millennium commences is to create systems by which such constructs, particularly pedicle-screw based constructs and interbody fusions, can be achieved utilizing less invasive techniques.

The implantation of pedicle screws, in and of themselves, using radiographic guidance and minimally-invasive techniques was easily achieved and the theoretic basis for such a technique was actually again described by the earlier pioneers including Roy-Camille, Wiltse, and Steffee. This has required some acceptance by the surgical community since it involves placing screws using fluoroscopic rather than direct visualization. This “Blind” technique, as referred to in some circles, required some re-education of surgeons, as well as a need for the surgical community to develop a “comfort” level in recognizing that such a procedure can be easily achieved with little risk to the neurologic or other structures.

The challenge to the implantation of such constructs has been to position the rods into the trailing ends of the screws, i.e. passing the rods through the soft tissues which lie in between the heads of the screws.

Pursuant of that trend, Justis, Sherman, and Foley introduced in U.S. Pat. No. 6,530,929—a technique wherein it was demonstrated that pedicle screws and a connecting rod can be placed using a minimally-invasive technique. This technique has now come into wide clinical use. One disadvantage of this system is that the method of passing the rod into position involves carrying the rod through soft tissues in the back which are remote from the target motion segment.

Such concepts have been furthered by Landry, who teaches yet another technique for the minimally-invasive implantation of pedicle screws and a stabilizing rod in U.S. Pat. No. 7,250,052 and U.S. Pat. No. 7,491,218. The embodiment of this system which is in clinical use involves creating a skin incision which is essentially the distance from the most cranial to the most caudal pedicle, and although such an incision is hardly considered significant in contrast to the incisions associated with “open” surgery, nevertheless this involves a larger incision than some surgeons (and patients) desire.

Boehm and Melnick demonstrated that a pivoting rod system could be useful in implanting a pedicle screw and rod system in U.S. Pat. No. 7,306,603. This has also been demonstrated to have wide clinical applicability. Again, in the form of this system currently on the market, a small incision is again created extending from the most cranial to the most caudal pedicle.

Other systems have been disclosed as well. These are all based on the implantation of the pedicle screws followed by dropping the leading end connecting rod into the trailing end of one screw, with the long axis of the rod aligned with the long axis of the screw. The trailing end of the rod is then manipulated such that the rod is rotated around the trailing end of the rod and passed into the other screw head or heads. There are multiple systems currently on the market that closely mimic each other using this technique. These systems are often reported to require significant manipulation in order to position the rod.

Therefore, systems for the implantation of pedicle screws utilizing minimally invasive techniques have been available for several years now. Yet, in all of these systems, there is are several persisting problems with the systems that are currently available including an element of disruption of tissues that are not directly related to the pathology, difficulties in seating the connecting rods, and the need for a securing bolt or nut to maintain the rod in position. Therefore, a need exists for a system which would accomplish implantation of a pedicle screw and connecting rod system which would involve less tissue disruption, provide greater facility in seating the rod, and avoid the use of locking elements, all contrast to those currently available and in clinical use. Such a system would be unique, useful, novel, and non-obvious.

### SUMMARY OF THE INVENTION

The principal object of the invention is to disclose a unique, useful, novel, and non-obvious system for implanting a pedicle-screw based spinal stabilization construct; the system is conceived of as being utilized principally as an adjunct to spinal fusion, using a minimally-invasive surgical technique. The invention addresses this object by providing a novel connecting rod to be utilized in conjunction with a novel expandable/retractable device. This device is specifically designed to create a tract through the paravertebral musculature and soft issues from the trailing end of a first [percutaneously-implanted] pedicle screw to the trailing end of at least a second [percutaneously-implanted] pedicle screw; said tract having been created, the novel connecting rod is then disposed therethrough by reversibly coupling the rod to the expanded device prior to retraction. In addition, also disclosed herein is a modification of the standard cradles found at the trailing ends of the pedicle screws, often referred to as the “Screw Heads,” which, in the current art, typically couple the rod or plate to the trailing ends of the screws in order to stabilize the construct. The modifications set forth herein are specifically designed to accommodate and couple the cradle at the trailing ends of the screws with the unique “engagement platform” which is provided to the connecting rods disclosed herein. In the preferred embodiment disclosed herein, the cradle is particularly unique inasmuch as the mechanism for securing the rods to the cradles is a self-contained mechanism which does not require the addition of any form of cap, nut, or bolt to secure the connecting rod to the trailing ends of the screws.

Additionally disclosed is a unique, novel, and non-obvious implantation cannula that provides for ideal placement of the screws using a minimally-invasive technique.

It should be noted that the techniques, pertinent clinical scenarios, and references to surgical techniques disclosed herein are primarily intended for use during percutaneous/minimally invasive surgical techniques. However, it should be borne in mind that such techniques and instrumentation could clearly be utilized during more traditional or “open” surgical procedures as well.

Hence, it is one object of this invention to disclose and provide such bone fastening pedicle screws that can be utilized in securing and stabilizing such a system. These screws are manufactured, in the preferred embodiment, from surgical grade titanium. Alternatively, such screws can be fabricated from stainless steel, various metal alloys, porcelain, various polyester composites, or any other substance known and/or acceptable to the art.

These screws are comprised of a leading end, a shaft, and a trailing end. The leading end is of sufficient sharpness and strength to primarily penetrate the bone of the pedicle, hence being classified as a “self-drilling, self-tapping screw.” However, an embodiment that would be best suited for implantation after drilling and/or tapping of the target pedicle can also be envisioned and is within the spirit and scope of the invention.

The shaft of the screws is of a standard configuration, including sharpness, pitch, and diameter and is provided with a ratio of inner diameter to outer diameter as has been determined to be most effective in the large body of science that has developed with respect to the state of the art of pedicle screw technology.

The trailingmost end of the shaft is enlarged into a [preferably] ovoid, hemisphere, sphere, multifaceted or any other substantially bulbous configuration. This enlargement irreversibly couples with the leading end of a cradle, said cradle having been provided to serve as the interface between the screw and the connecting rod. It is anticipated that in the preferred embodiment, the enlarged trailingmost end of the screw is encased within a chamber provided within the leading end of the cradle. This chamber generally recapitulates the configuration of the enlarged trailingmost end of the screw, but is slightly larger and, therefore, can create a moment of rotation of the cradle around the enlarged trailing end of the screw. Hence, by providing this moment of rotation, the articulation between the trailing end of the screw and the cradle provides the cradle with some flexibility with respect to reconciling the angle of the screw placement (dictated by the angle of the pedicles, which may vary from vertebra to vertebra) with coupling the elongated rod element to the cradles of multiple levels.

This cradle, which is functionally the trailing end of the screws, is provided with a unique feature which is configured such that it results in coupling the screws to the rod, specifically by the coupling of the cradle to the terminal portions, or “engagement platforms” found at the lateral ends of the connecting rod.

The cradle, in the preferred embodiment, is a monolithic frame which is irreversibly coupled to two structures: at its leading end, the cradle couples with the trailingmost end of the shaft of the screw.

The trailing end of the cradle couples with the leading end of a rotatable cam-shaft-disc complex. This complex is ultimately the actuator mechanism by which the cradle secures the rod in place.

The frame of the cradle, as viewed in the frontal view, very generally assumes an “H”-shaped configuration. The bridging component of the “H” configuration is referred to hereinafter as the base of the cradle, and is orthogonal to the long axis of the bone fastening screws. The base of the cradle creates a central reference point in describing the frame inasmuch therefrom the cradle is provided with a superior channel and an inferior chamber: the superior channel is that component which is found above the base, as viewed in the frontal view, and shall ultimately serve as the capture surface for the engagement platforms of the connecting rod; the inferior chamber, below the base as viewed in the frontal view, comprises the leading end of the frame of the cradle and hence, that aspect of the frame which articulates with the trailingmost end of the shaft of the screw. It can be recognized in the frontal view that the superior channel is created by two projections which are monolithic with the base and extend superiorly (in the frontal view). These form the medial and lateral walls of this channel.

It is anticipated that in the preferred embodiment, the frame will be manufactured from titanium. Certainly surgical-grade stainless steel could also be used, as well as any other metal such as nickel, molybdenum, or any alloy; additionally, porcelain, as well as plastic or polyester substrates may be used. Any substance known or acceptable to the art could be utilized in this fashion to manufacture the frame.

The medial and lateral walls creating the central channel are, in the preferred embodiment, configured such that the trailingmost aspect of these walls—those aspects of the walls that are farthest from the base, are symmetrically attenuated when contrasted to those aspects of these walls that are found immediately arising from and in close proximity to the base, which are substantially greater in thickness, Furthermore, these walls can be described as having an interior surface and an exterior surface. The interior surface is that aspect of the walls which form the surface of the central channel, while the exterior surface is the outside of the cradle. The exterior surface is continuous with the outer surface of the rest of the cradle, while the variances of the wall thickness described above are reflected by variations of the interior surface.

Hence, this configuration creates a wider channel known hereinafter as the rod channel in the trailingmost aspect of the cradle when compared to that aspect of the channel found in the immediate proximity to the base of the frame of the cradle, wherein a narrower channel is found.

The rod channel found at the trailingmost aspect of the cradle is specifically designed to accommodate the central expanse of the rod channel (which is wider as viewed from the frontal and top views than the engagement platforms), as well as the rod positioner (to be described below), which is the actuator that transfers the leading end of the rod from one cradle to the other cradle. These rod channels are specifically dimensioned such that during manipulation/positioning of the rod the central portion of the rod doesn't engage the narrower channel of the cradle.

The narrower channel which is closer to the base of the cradle accommodates an engagement platform of the rod, and shall hereinafter be referred to as the capture surface of the cradle.

Additionally, the cradle frame is also irreversibly coupled with a locking cam complex, this complex being critical to the coupling of the cradle with the rod. In the preferred embodiment, this locking cam complex is a monolithic structure comprised of a cam which is then continuous with a shaft which is, in turn, continuous with a rotatable disc-like base. Alternatively, the cam, the shaft as well as the disc could be separate components which are irreversibly coupled, and the coupling of any combination of these elements is also conceivable. This cam/shaft/rotating disc complex has already been referred to and is hereinafter known as the cam-shaft-disc complex.

This structure is, in turn, irreversibly coupled with the frame of the cradle. Specifically, the base of the cradle is coupled with this disc element of this complex. As specified above, the cam is mounted on a shaft of varying lengths. Principally, the length of the shaft must be greater than the thickness of the engagement platform and the base of the cradle to achieve the desired effect. This cam may be semicircular in configuration, or may be of any configuration that is biomechanically sound and achieves the goals to be outlined directly below.

The shaft, in turn, passes through an aperture at the base of the cradle, being ultimately monolithic with or irreversibly coupled to the disc. The disc is contained within the frame of the cradle, this disc being placed within a chamber of a similar but slightly larger configuration; this allows the disc to rotate freely, and in that fashion provides rotation to the shaft and ultimately to the cam. As previously disclosed, the rotation of the cam within the frame of the cradle is the actuator mechanism that is ultimately responsible for the method by which the rod is secured to the cradle (and thereby the screws).

Recalling that the frame is then irreversibly coupled at its leading end with the trailing end of the shaft of the bone fastening screw, it is envisioned that in the preferred embodiment, the leading most end of the cradle is provided with an aperture through which the trailing end of the shaft of the screw passes therethrough. This aperture is the same configuration (presumably round) as the shaft, and is slightly larger in diameter than the shaft, in order to accommodate a moment of rotation provided to the cradle around the trailing end of the screw.

As previously disclosed, the expanded trailingmost end of the shaft of the screw is encased within the inferior chamber which recapitulates its configuration but is slightly larger, creating a moment of rotation of the cradle around the trailing end of the screw. Specifically, this geometric configuration permits the vertical axis of the cradle to vary slightly from the angle of the long axis of the screw so as to accommodate positioning of the rod in the setting where the target pedicles to be stabilized differ in their trajectories from one vertebra to the adjacent vertebra, thus requiring that the screws be implanted at slightly different angles. By providing some flexibility in positioning the cradles, these varied angles can be reconciled with and securing the connecting the rods to the cradles can be facilitated in the setting whereby the angles of the pedicles are varied.

It is to be further appreciated that, as already established, within the base of the cradle is found the rotating disc as well as the trailing end of the shaft of the screw. Ideally, both structures are contained within a common chamber; alternatively, if these two structures are contained within separate chambers, there must be at least a communication between the two chambers which would then facilitate reversible coupling of the trailingmost end of the screw and the leading end of the rotating disc.

Providing a mechanism whereby the cam-shaft-disc complex reversibly fixes the position of the screw, cradle, and complex with respect to one another is critical to the implantation of the screw within a target pedicle as well as locking the rods in place once they have been positioned.

Therefore, it is imperative that the leading end of the rotating disc closely relate to the trailingmost end of the screw. This is because when the cam-disc complex is deployed in securing the engagement platform, the cam is displaced against the capture surface of the cradle. Through this action, the leading end of the disc will reversibly couple with the trailing end of the shaft of the screw. This coupling may be accomplished through a variety of embodiments. It can be accomplished by providing the leading end of the rotatable disc with a configuration which would mate with an appropriate area in the trailingmost end of the hemisphere or sphere. It is anticipated that in the preferred embodiment, this is accomplished by providing the leading end of the disc with a spherical protrusion, and providing the trailingmost end of the screw with an associated concavity. In doing so, the spherical enlargement could engage the concavity if the long axis of the cradle is aligned with the long axis of the screw, but with this configuration the enlargement would also engage the screw if there was a slight variance between the cradle and the screw. Obviously, such a result could also be achieved with a Hex head, but this might require greater alignment of the cradle and screw. However, any other similar embodiment that would reversibly couple the cam to the trailingmost end of the screw would be within the spirit and scope of the invention. The critical aspect of engaging the leading end of the disc with the trailingmost end of the screw is that the [reversible] coupling of these two structures results in elimination of any rotation of the cradle around the trailing end of the screw. Any such continued movement would be counterproductive to the goal of the invention to stabilize the target motion segment.

This coupling mechanism, in turn, participates in the fashion by which these screws are implanted. Once these elements have all been fixed in position with respect to one another, the entire screw and cradle complex can be rotated in unison by the device which inserts the screw into the target pedicle (to be disclosed below).

Upon implantation of the screws, the cams will be found to be in the non-deployed position, specifically such that the long axis of the cam will be parallel to the greatest dimension of the apertures found in the engagement platforms of the rods. Given this, upon transfer by the actuator of the rod from the secondary screw to the primary screw, the apertures in the engagement platforms are centered over the rotatable cams. A purpose-specific instrument, which will also be disclosed in further detail, is then utilized, engaging the cam while displacing and thereby pressing the engagement platform of the rod against the capture surface of the cradle. Once the platform has been pressed against the capture surface of the cradle, the cam has been passed through the aperture in the engagement platform; at that point, the purpose-specific instrument will rotate the cam approximately one-quarter (90°) turn. With this rotation of the cam, the greater dimension of the cam is transverse to the long axis of the rod, and therefore transverse to the frame creating the aperture of the engagement platform. Upon completing this action, the cam will become locked against the walls of the channel of the cradle. Moreover, the cam has secured the engagement platform against the capture surface of the cradle, in that way securing the rod in place. In the ideal embodiment, the configuration of the walls of the frame of the cradle exists such that as the cam is rotated, the cam-disc complex is slightly displaced towards the trailingmost end of the screw, the cam being captured within the walls of the cradle by a specific configuration of the walls of the cradle.

This specific configuration of the superior channel is best appreciated in the frontal view. When viewed from that perspective, it can be seen that this channel assumes a substantially “hourglass” configuration inasmuch that the frontal profile of the channel reveals there to be symmetric protrusions of the interior surfaces of the medial and lateral walls leading to a narrowing of the channel approximately mid-position between the base and the trailingmost ends of the medial and lateral walls. This narrowing is intended to permit positioning of the engagement platform against the base of the cradle; however, the narrowing is also intended to prevent the central expanse of the rod or the rod insertion device from being inadvertently positioned in the base of the cradle. The lower half of the “hourglass” configuration, furthermore, is configured such that it permits free rotation of the cam when there is no engagement platform in place (during insertion of the bone fastening screw into the pedicle), but when the engagement platform is in position against the capture surface of the cradle, then a 90° rotation of the cam will result in compression of the cam against the walls of the cradle, and, furthermore, compel the leading end of the cam-disc complex (it has already been disclosed that the leading end of the disc is configured to reversibly couple with the trailing end of the shaft of the screw) to be driven towards the trailing end of the screw, engaging and “locking” with the trailing end of the screw, thus eliminating any further rotation and in effect further “locking” the construct in place.

In addition, in the preferred embodiment, there is another unique element provided to the inner surface/central channel of the cradle. Each of the side walls of the lower half of the “hourglass” are provided with a single crenellation which is strategically positioned so as to act as a ratcheting/locking mechanism when the cam is rotated and deployed. This is to prevent inadvertent de-rotation of the cam with loosening and disengagement of the rod, which would be most undesired.

However, it must be recognized that in some instances, the rods and even at times the screws require removal. To that end, a device is disclosed which will be described in greater detail below but which will effect removal of the rods be slight displacement of the medial and lateral walls of the central channel while de-rotating the cam. In this fashion, the rods can be uncoupled from the trailing ends of the cradles.

This rod-cradle coupling can, of course, only be understood by also disclosing a complete description of the connecting rod element. The connecting rod element, in the preferred embodiment, is an elongated element which is provided with a leading end, a central element, and a trailing end. The preferred embodiment, as viewed from the lateral perspective, is somewhat standard in appearance when compared to other existing art, being slightly curvilinear in configuration with the convex aspect directed anteriorly towards the patient's body (when in final position) and the concave aspect directed posteriorly. This curvilinear configuration provides two advantages: firstly, it encourages the construct to assume a configuration consistent with the natural lordosis of the spine, and in particular the lumbar spine; secondly, it provides a configuration which is more easily manipulated and positioned using a minimally-invasive technique as compared to a straight rod. However, a straight rod could be utilized as well and certainly complies with the spirit and scope of the invention. A rod with a curvilinear profile in the direction opposite of that defined above would have no particular advantage in the lumbar spine but would be consistent with the natural lordosis of the thoracic spine and for that reason is also included as a potential embodiment which is again within the spirit and scope of the invention. As viewed in cross section, the rod would appear round or slightly ovoid. It is anticipated that the rod would be composed of titanium, in the preferred embodiment, although stainless steel, steel-nickel alloy, any other metal, porcelain, composite, any polyester, or any other substance known and/or acceptable to the art could also be utilized in the manufacturing of this element.

The unique aspects of the rod are the leading and trailing ends, or terminal portions, which, in the preferred embodiment are identical “mirror images” of each other. These terminal portions are represented by a flattened, horizontal extension which serves as an engagement platform, and shall hereinafter be thusly known. This flattened expanse extends in the long axis of the rod with the length of the engagement platform being its greatest dimension. It is somewhat smaller in width than the diameter of the rod. The height of the engagement platform is its smallest dimension. It can be appreciated from the forgoing description that this engagement platform is, in the preferred embodiment, somewhat rectangular in configuration. These platforms are oriented such that the narrow aspects of the platforms are seen viewed from the lateral view, These flattened extensions are configured so that in the fmal position, most critically, these platforms must be substantially orthogonal to the long axis of the screws; this orientation would thereby position the engagement platforms to be coupled to the cradle which is, in turn, coupled to the trailing end of the screw.

A key element of the configuration of the engagement platforms is the presence of a central aperture which is provided to the central portion of the horizontal expanse. This aspect of the platform is best seen from the top view. These apertures serve as the site whereby the cradles are coupled to the connecting rods. Therefore, as mentioned above, it is critical that the engagement platforms are configured and oriented in a fashion that they are easily positioned within the cradle.

It is anticipated that in the preferred embodiments, these engagement platforms are either rectangular in shape, or somewhat ovoid or semi-ovoidal in configuration. The central apertures in these embodiments will generally closely mimic the configuration of the engagement platform itself. The role of the engagement platforms as they relate to the cam-shaft-disc mechanism has already been thoroughly reviewed above.

An important alternative embodiment of this mechanism must be disclosed. In this alternative embodiment, the engagement platforms are not provided with an aperture. Rather, the engagement platforms are provided with the cam-shaft-disc mechanism complex (discussed above and completely disclosed below), which is positioned such that the cam and shaft are directed towards the cradle. It can be envisioned that this complex, in the non-deployed position, is positioned at the end of the rod in such a way that it easily articulates with the rod positioner (to be disclosed below). It can be further envisioned that in such an embodiment, the capture surface of the cradle is provided with an aperture into which the cam may be inserted.

Once positioned against the capture surface of the cradle, the complex may be deployed, creating a locking mechanism to hold the rod in place, as well as locking the cradle against the trailing end of the shaft of the pedicle screw, This would be accomplished because in such an embodiment, upon deployment of the cam-shaft-disc complex, the cam would be displaced into this aperture in the capture surface of the cradle. Once the cam is displaced into the aperture in the capture surface of the cradle, the cam is rotated in a fashion similar to that described above, thus locking the rod into place. Furthermore, in this alternative embodiment the frame of the cradle will be configured in such a fashion that the leading end of the cam will engage the trailing end of the shaft of the screw, and in that way lock the cradle against the trailing end of the screw. This would provide further stabilization to the entire construct.

In order to further appreciate the invention in its unique, novel and nonobvious approach to the solution of the shortcomings of the current art, it must be appreciated that each of these bone fastening screws are implanted by being passed through a working cannula; these cannulae remain in place and are utilized for positioning the rod in place as well.

Therefore, herein disclosed is a unique insertion cannula which is designed to facilitate the percutaneous placement of the pedicle screws. The challenge of this component is to accurately identify and engage certain specified subsurface structures while using localization methods other than direct visualization to achieve the surgical goals. This object is achieved by providing a cannula-trocar complex which is unique inasmuch that the trocar and cannula are composed of radiolucent material, except for the leadingmost ends. The trocar, known for the purposes of this application as the guidance trocar, is provided with a leading end, a shaft, and a trailing end; the leading end is designed to initially penetrate the bone at the base of the pedicle. This component of the trocar is, in the preferred embodiment, composed of any metal known or acceptable to the art. This leading end is of sufficient sharpness and strength to penetrate the cortical bone of the entry point to the pedicle. Because this is the only portion of the system which is radiopaque, placement of this leading end into the pedicle by any form of intraoperative imaging that utilizes X-rays (including X-rays, fluoroscopy, CAT scanning) is facilitated.

One unique aspect of the guidance trocar is that in the preferred embodiment, the guidance trocar is tapered to a fine point at the leading end, which assists in passing this instrument through the soft tissues as well as manipulation and repositioning of the leading end; however the shaft and trailing end of the trocar are widened to the point that the outer diameter of the trocar is just slightly smaller in diameter than the inner diameter of the insertion cannula. Handgrips may be provided at the widened trailing end of the trocar to facilitate manipulation of the trocar during insertion. Alternatively, a detachable handle may be provided. Other embodiments of the shaft may be envisioned by those skilled in the art; such embodiments are, of course, all included in the spirit and scope of this invention.

One important consideration in providing the trocar-cannula complex involves the leading end of the trocar along which the diameter of the tapered segment is significantly less than the diameter of the insertion cannula. In the preferred embodiment, the tapered segment is quite acute such that the transition from the tapered segment to the full diameter represents a fairly small segment of the long axis of the instrument, essentially creating a “hub” at the transition point.

It is anticipated that in such an embodiment, the tapered segment will be inserted completely into the pedicle, essentially being passed to the “hub.” In such an embodiment, the insertion cannula is then passed over the outer surface of the trocar without the possibility of incarcerating tissue within the insertion cannula.

However, if the tapered segment is more gradual, then a potential space can be created between the outer surface of the leading end of the shaft of the trocar and the inner surface of the insertion cannula. The problem that such a potential space creates is that when the insertion cannula is positioned into place, soft tissue can be trapped between the outer surface of the trocar and the inner surface of the insertion cannula, thus possibly incarcerating these tissues within the cannula. This, in turn, could lead to difficulties in executing the remainder of the procedure.

An alternative embodiment is disclosed to speak to this issue. In this alternative embodiment, the leading end of the trocar is expandable. This is accomplished by either providing; in one instance, an expandable an expandable membrane on the outer surface of the trocar, which can be expanded either hydraulically or pneumatically; or in a second embodiment, a mechanically expandable shaft. In either instance, such an embodiment would result in eliminating the potential space and preventing incarceration of the soft tissues.

The insertion cannula which is utilized to insert the primary screw is unique in its configuration. The primary screw is that screw which is inserted in anticipation of accommodating the expandable rod positioner. Because this device must be accommodated through the primary cannula, the configuration of the cannula must account for and accommodate the passage of said device.

This would result in additional requirements upon the cannula beyond merely permitting and accommodating the passage of the screw/screw insertion complex. The cannula must also accommodate the expandable rod positioning device. This device must be positioned somewhat eccentrically with respect to the main channel of the insertion cannula, so that upon retraction of the expandable rod positioner during the process of positioning the leading end of the connecting rod within the cradle of the primary screw, there is access granted to the cam deployment apparatus so that it may couple the leading end of the connecting rod with the cradle of the primary screw. Therefore, the expandable rod positioning device must be positioned either cranial or caudal to the main channel of the cannula. This object is achieved through one of three principle configurations.

Firstly, the cannula may be somewhat irregularly ovoid in configuration as viewed from the top view, with the ovoid configuration specifically designed for the purpose of accommodating passage of the expandable rod positioning device.

A second configuration provides for a removable panel which is found along that aspect of the cannula which must accommodate passage of the expandable rod positioning device. At the appropriate time during the surgical intervention, the panel is removed and the insertion device is positioned.

A third configuration provides part of the perimeter of the cannula to be comprised of an elastomeric substance which can then be expanded during that portion of the insertion of the construct which requires utilization of the rod positioning device.

Hence, the patient is positioned on the operating table, presumably in the prone position, and adequate anesthesia is accomplished. Intraoperative imaging must be utilized, with virtually any form of intraoperative imaging being acceptable. This would include X-rays, fluoroscopy, CAT scanning, any of the “frameless” stereotactic systems and image-guided surgical systems now available and known to the art, or intraoperative MRI scanning. It is anticipated that intraoperative MRI scanning is not ideal, given the amount of artifact that would be generated by the metallic screws. Porcelain screws, or screws composed of extremely durable composite plastics (such as extremely durable nylon) would create less artifact on MRI scanning, but have not yet achieved widespread use in the known art. The use of intraoperative MRI would also obviate the design of the trocar cannula complex, which as stated above are radiolucent except for their leading ends.

Once the pedicles of the target motion segment or segments have been identified, the trocar is passed into the primary pedicle through a small stab incision in the skin. The tapered segment allows for the trocar to be passed through the soft tissues using a gentle rotating motion. Once the trocar can be felt against the entry point of the pedicle, image-confirmation of the position is obtained, and the trocar is passed into the pedicle, The primary cannula is then passed over the trocar which is stabilized by its insertion into the bone. The trocar is then removed in preparation for the implantation of the primary screw.

Unlike the trocar, which is stabilized and prevented from shifting positions because the leading end of the trocar is inserted into the bone, the cannula must be stabilized so that the tract initially developed by the trocar can be utilized to insert the screw proper.

At that point, another unique aspect of the cannula is deployed. The cannula is provided with a leading end which is radiopaque, and which is provided with a plurality of sharpened points at the leading end. This permits the cannula to be stabilized against the entry point of the pedicle, Additionally, the cannula may be provided with a deployable fin which would stabilize the cannula against the facet joints. The guidance trocar is then removed, and the cannula—with its radiolucent shaft and stabilized radiopaque leading end, serves as a guide to screw placement as well as a working cannula for the remainder of the procedure. In the preferred embodiment, the radiopaque leading end literally serves as a “Bull's Eye,” for aiming the insertion of the screws.

At that point, the primary screw in implanted. As previously disclosed, the screws may be either “self-drilling, self-tapping,” in their design, or a drill and/or a tap may be utilized in creating the hole in the pedicles sufficient to accommodate the screw.

A screw insertion apparatus is now disclosed which is necessary for the insertion of the screws. This device must speak to several challenges.

Firstly, as one of the primary objects of the invention is to disclose a device which requires a minimally-invasive procedure, it is most desirable to provide a cannula of the smallest possible dimension. The diameter of the cannula is only slightly larger than the diameter of the cradle of the screw, which is the largest part of the screw complex. Therefore, it would not be desirable to create an insertion apparatus which is fitted around the outer diameter of the cradle, but rather the preferred embodiment of such an apparatus would have a leading end which would engage within the central channel of the cradle.

Secondly, the cradle has been provided, as discussed above, with a moment of rotation around the trailing end of the screw. Unfortunately, that same moment of rotation will destabilize the cradle-screw complex during insertion unless the insertion device is provided with a mechanism by which the complex is stabilized.

As discussed and disclosed above, the moment of rotation can be eliminated by locking the leading end of the cam-shaft-disc complex (specifically the leading end of the disc) against the trailingmost end of the screw proper. Therefore this insertion apparatus must have a leading end configured to accomplish these goals.

This can be best accomplished by providing a device which in its most general configuration has a leading end, a shaft, and a trailing end, and is uniquely designed to meet the challenges disclosed above insofar that it has been provided with an inner and outer shaft. The trailing end of the device is that which interfaces with and is controlled by the surgeon during the surgical implantation of the system. The shaft is of sufficient length so that the device may be inserted through the cannula in such a fashion so that the surgeon may manipulate the activities of the leading end by interface with the trailing end.

The outer shaft is configured such that the leading end has a profile in the frontal view that closely recapitulates the profile of the cradle, such that the leading end of the outer shaft can be reversibly coupled with the central channel of the cradle in a substantially secure fashion. Once this is accomplished, the inner shaft is then deployed. The leading end of the inner shaft is configured so as to reversibly couple with the cam. Furthermore, once this coupling has been achieved, the inner shaft is displaced toward the base of the cradle with respect to the position of the outer shaft so that the cam-shaft-disc complex is displaced and compelled to couple with the trailingmost end of the screw, allowing the screw and the cam-shaft-disc complex to rotate as a unit, with the cradle remaining immobile as it is secured against the outer shaft of the screw insertion device. In this fashion, by rotating the cam-shaft-disc complex, the screw is rotated and hence inserted into the pedicle.

The invention accomplishes positioning of the elongated element or connecting rod with minimal disruption of the intervening tissues owing to the use of a unique, purpose-specific implantation device, known hereinafter as the expandable rod positioner, which is found at the leading end of a device known as the connecting rod placement device. In the preferred embodiment, this device is inserted into the insertion cannula, and is provided with a leading end, a shaft, and a trailing end. The leading end of the device, or expandable rod positioner, is provided with an “accordion like structure,” which is provided with an outer sheath composed of flexible material which in the non-deployed position demonstrates multiple folds which comprise the “accordion” and which are maximally folded and compressed together. As the device is expanded, the folds comprising the “accordion” become maximally “unfolded,” thus allowing the device to expand in a craniocaudal dimension—which is the dimension between the pedicles of adjacent vertebrae of the spine. Within this mechanism is a central core composed of a flexible material. The flexible core extends from a trailing end which passes out of the working cannula and can be manipulated by the surgeon. As the flexible core extends down the working cannula, it assumes a near right-angle turn prior to the point at which the leading end of the flexible core becomes encased in the accordion sheath. When the trailing end of the flexible material is displaced by the surgeon towards the working cannula, the leading end along with its encased “accordion” sheath will be displaced towards the other screw and the leading end of the connecting rod. This actuator is found at the leading end of a device known as the connecting rod placement device.

Accordingly, in the preferred embodiment, the leading end of the expandable rod positioner is provided with a pair of jaws that can be manipulated by the surgeon and which are specifically designed to be deployed and in doing so capture the leading end of the connecting rod; that having been accomplished, the connecting rod can be retracted back towards the cradle of the primary screw. In that fashion, the rod is positioned connecting the trailing ends of the pedicle screws, with the positioning of the rod occurring with minimal disruption of the associated soft tissues. Of course, those skilled in the art can envision yet other embodiments which would allow for an expandable/retractable element which can achieve the goals and objects of the invention, and such embodiments would also lie within the spirit and scope of the invention.

It is also necessary to disclose the insertion cannula of the secondary screw, which initially delivers the rod for presentation to the expandable rod positioner. It is envisioned that this insertion cannula is also uniquely configured, specifically with an expansion being positioned on the side of the cannula opposite the side which is adjacent to the insertion cannula of the primary screw. This is best understood by studying the example of an L4-L5 construct, in which the primary screw is L5. In such an example the expansion would be located on the cranial side of the insertion cannula of the L4 screw.

This geometry is provided so as to position the rod on a bias, with the leading end protruding into the soft tissues between the cradles, and therefore in a fashion that favors negotiating the rod into final position with the use of the expandable rod positioner. The extension to the side of the cannula accommodates the trailing end of the rod when it is positioned in readiness for the deployment of the positioner. If the rod were positioned along the vertical axis of the insertion cannula without providing this additional bias, the rod may not easily negotiate the orthogonal angle at the trailing end of the cradle.

After the insertion of the secondary screw, the rod is placed into the secondary cannula with the use of a rod holder. This is an instrument which is also provided with a leading end, a shaft, and a trailing end; the leading end grasps the connecting rod, specifically grasping it at the trailing end. This stabilizes the rod within the central channel of the cannula, and also provides the surgeon with a means of pushing the rod into the tract between the cradles and in that assuring that the expandable rod positioner can engage the leading end of the rod.

The aforementioned jaws are, in the non-deployed position, resting against one another and forming a substantially semi-ovoid or “bullet” configuration. They protrude from the leading end of the expandable rod positioner in a manner such that this configuration contributes to creating a path through the soft tissues that exist between the two cradles found at the trailing ends of the screws. As previously stated, this expandable rod positioner is expanded by manipulation of the trailing end of the flexible core by the surgeon, specifically with displacement of the trailing end. This flexible core is also provided with a series of cables which extend from the trailing end of the flexible core to the trailing ends of the jaws found at the leading end of the expandable rod positioner. Furthermore, at the interface between the cables and the jaws there is an articulation of the trailing ends of the two jaws with a piston or rod-like axis which is positioned transversely across the leading end of the flexible core. The articulation creates a hinge-like relationship between the trailing ends of the jaws and the axis such that when the cable system is deployed, the trailing ends of the jaws will rotate around the hinge, thus resulting in an “opening” or separation of the leading ends of the jaws, permitting capture of the leading end of the engagement platform found at the leading end of the connecting rod. Once the engagement platform (and hence the rod) is captured by the jaws, the jaws are then closed; it can be envisioned that a spring may be positioned (in at least one embodiment) contributing to closing of the jaws.

The flexible core is then retracted by the surgeon, resulting in displacement of the leading end of the rod from the secondary screw-cradle complex to the primary screw-cradle complex, at which point the jaw mechanism is again deployed, resulting in disengagement of the jaws from the leading end of the engagement platform of the connecting rod. It is anticipated that in order to expedite this component of the procedure, the rod positioning device is removed simultaneously as the jaws are opened; in this fashion, the insertion cannula will encourage release of the jaws from the leading end of the rod.

Other mechanisms by which the leading end of the expandable rod positioner can capture the leading end of the connecting rod can also be envisioned by those skilled in the art. Such mechanisms would include a magnetically-driven mechanism as well as an hydraulically or pneumatically driven mechanism for expanding and releasing the jaws which couple with the leading end of the connecting rod. Such embodiments would be considered to be within the spirit and scope of the invention.

Additionally, any hydraulically or pneumatically-driven mechanism by which the expandable rod positioner can be deployed and disengaged is also conceivable and can be envisioned by those skilled in the art. In such a mechanism, the folds of the “accordion,” are represented by a series of bellows which can be expanded and contracted, thus accomplishing the goals of the device.

Broadly use of the claimed apparatus is by the method of comprising the steps of: positioning the patient on the operating table, preferably in the prone position, identifying the target pedicles using fluoroscopy, intraoperative x-rays, intraoperative CAT scanning, intraoperative MRI scanning, or any other image-guided technology; creating a sterile field through which the surgical procedure can be performed; for each target pedicle, inserting an initial, purpose-specific guide trocar; affirming the position of the leading end of the guide trocar within the entrance to the base of the pedicle; passing a purpose-specific working cannula over each guide trocar while removing the guide trocar; securing each working cannula to the bone; passing a drill through each working cannula into the target pedicle associated with each working cannula; passing a tap through each working cannula; inserting a primary screw into at least one pedicle on each side; inserting a secondary screw into a second pedicle on each side; ascertaining that the markers on the screw insertion devices indicate proper alignment of the primary and secondary screws with respect to each other; distracting the cannulae, using the purpose-specific distractor so as to restore the intervertebral height to a desired dimension; using the calibration on the distractor to determine the appropriate sized rod; removing the screw insertion devices and then inserting the expandable rod positioning device into the cannula associated with the primary screw; inserting the rod cartridge into the cannula associated with the secondary screw; deploying the expandable rod positioner so as to create a tract in the soft tissues between the primary and secondary screws; capturing the leading end of the connecting rod within the leading end of the expandable rod positioner; returning the expandable rod positioner to the non-deployed position thus into the cannula associated with the primary screw and thus displacing the rod from the trailing end of the secondary screw and compelling the rod to be re-oriented so that the leading end of the rod is now encased within the. trailing end of the primary screw while the trailing end of the rod is encased within the trailing end of the secondary screw; locking the leading and trailing ends of the rod within the trailing ends of the primary screw and secondary screws; removing all cannulae; closing the incisions.

Once the rod has been positioned, the cam mechanism must then be deployed in order to secure or “lock” the rod in place. There is a purpose-specific device that accomplishes this goal, hereinafter known as the cam locking apparatus. In a fashion similar to the screw insertion apparatus, this device is provided with a leading end, a shaft, and a trailing end and is also provided with an inner and outer shaft elements. Each of these is provided with a trailing end which allows the surgeon to manipulate the leading end. Each is provided with a shaft, and a configuration is disclosed by which the inner shaft passes through the outer shaft. The outer diameter of the inner shaft is slightly smaller than the inner diameter of the outer shaft thus permitting the inner shaft to rotate freely within the outer shaft. The principal difference between these two elements is that the leading end of the outer shaft is designed to stabilize the cradle by reversibly coupling with the trailingmost end of the cradle, which is the slightly wider aspect of the central channel of the cradle as seen in the frontal view. This is best accomplished by providing the leading end of the outer shaft with a spherical or ovoidal configuration. It can be recalled from the discussion above that the trailingmost end of the cradle is meant to accommodate the rod as well as the expandable rod positioner, and hence a configuration that is rounded will most readily couple with this aspect of the cradle.

Conversely, the inner shaft is configured so as to couple with the cam. Once this reversible coupling is complete, the inner shaft rotates the cam so as to couple it with the walls of the cradle, while depressing the cam-shaft-disc complex so as to eliminate the moment of rotation around the trailing end of the screw. This is accomplished at all of the levels of the construct and the implantation is complete.

Yet another disclosure is of an instrument designed to disengage the cam from the cradle and in that way free the rod for removal, which may be necessary for a variety of reasons including discomfort associated with the hardware and infection.

This device would also be configured so as to be effective in the setting of a minimally invasive procedure; for that reason, this device would also be provided with a leading end, a shaft, and a trailing end. The leading or functional end would be configured so as to engage the cradle/rod complex. The shaft is designed to provide access in a minimally invasive setting, and the trailing end is provided with a handle by which the device interfaces with the surgeon.

The leading end is provided with a configuration which can mate with the cam, as well as an element which can interface with the walls of the cradle. These actions must be executed independently and simultaneously, and for that reason the device shall be provided with dual elements throughout including leading end, shaft and trailing end. Given that configuration, it can be envisioned that the device engages the target structures, and initially the element disposed to interacting with the walls of the cradle is deployed. This causes the walls to be slightly distracted away from each other, thus disengaging the ratcheting mechanism which serves to “lock” the cam in place. Once this is accomplished, the cam can be rotated into the original position and the rod removed.

### ALTERNATIVE EMBODIMENTS

Alternative embodiments for maintaining the cannula in position are also provided. Of course, one such embodiment is to secure a surgical stabilizing arm to the operating table and couple the trailing end of the cannula to the stabilizing arm. Such technologies have been utilized for more than two decades and are well-established in current surgical arts.

Another embodiment involves a unique configuration of the leading end of the cannula, in which there is a plurality of deployable arms provided to the outer surface of the cannula. At least two of these extend out in such a fashion that they extend over the superior and inferior aspects of the base of the transverse process, while at least a third deployable arm will lock against the lateral aspect of the inferior articular process. This fixation of at least three points will reduce the chances of migration of the cannula during insertion of the screw.

In another embodiment, a plurality of electromagnetic needle probes are positioned via separate puncture entry points around the cannula, which is provided with magnetic elements positioned along the long axis of the cannula. By activation of the probes, and proper balance of the magnetic elements, the cannula will be held in place.

An alternative embodiment of the cannula associated with placement of the secondary screw can also be envisioned. As mentioned above, a concern is negotiating the rod through an essentially orthogonal angle at the trialing most end of the screw. This can be addressed by providing any embodiment which permits the rod to achieve a greater bias than being aligned with the long axis of the screw.

In one embodiment, the cannula utilized for insertion of the screw is essentially round as viewed in the top view. The rod, along with a flexible rod pusher/holder are all loaded within a cartridge, which in turn can be reversibly coupled with the wall of the channel. The cannula has been specifically designed such that there is an aperture at its leading through which the leading end of the rod may be pushed once the cartridge is loaded.

Alternative embodiments of the actuator mechanism responsible for positioning of the connecting rod can be envisioned. One alternative embodiment provides for an expandable/retractable rod positioner is a series of concentric cylinders lying in sequence within each other, and is known as the telescoping rod positioner. As in the case of the expandable rod positioner, the telescoping rod positioner is found at the leading end of a device known as the connecting rod placement device.

This telescoping rod positioner is designed to be contracted in the non-deployed position, and with deployment, expand such that the leadingmost end of the set of cylinders extends from the trailing end of a primary screw to the trailing end of a secondary screw. The leadingmost end of the telescoping rod positioner is configured so as to reversibly couple with the leading end of the connecting rod which has been positioned within the trailing end of the secondary screw. With deployment of the device, the telescoping rod positioner becomes fully expanded, and in that expansion the device passes through the intervening tissues between the trailing ends of the two screws. Once coupling between the leading end of the connecting rod and the leading end of the rod positioner has occurred, the telescoping rod positioner is retracted to the non-deployed position. In doing so, it carries the leading end of the connecting rod back through the tract in the paravertebral soft tissues and into the trailing end of the primary screw. Thus, in that fashion, the telescoping rod positioner serves as the actuator positioning the connecting rod into the trailing ends of the pedicle screws.

As mentioned, the telescoping rod positioner is comprised of a series of concentric cylinders; a feature of these cylinders is that in each pairing of cylinders, in which an inner cylinder is contained within an outer cylinder, there is a special configuration such that in the non-deployed position, the inner cylinder is contained entirely, or at least nearly entirely, within the outer cylinder. This is accomplished because each cylinder within the series is provided with an outer diameter which is just slightly smaller than the inner diameter of the next largest cylinder. Hence, such an arrangement would be referred to as a “pairing” of cylinders, with an “inner” cylinder being contained within an “outer” cylinder; any number of cylinders can, therefore, be positioned within one another in such a fashion.

The total number of cylinders within the sequence is therefore dependent primarily on two factors: firstly, the distance that the entire sequence must traverse in the deployed position, and secondly, the length of each cylinder from the leading end of the cylinder to the trailing end of the cylinder.

Another critical feature of this series of cylinders, or telescoping rod positioner, is that the inner diameter of the leading end of each cylinder has been provided with an additional thickness or lip, in the preferred embodiment. Furthermore, the trailing end of each cylinder is also provided with a flaring or enlargement such that this flaring will interface with the increase in the inner diameter of the larger cylinder. Together, this mechanism serves to prevent the inner cylinder from being displaced out of the outer cylinder during deployment.

A number of mechanisms by which the telescoping rod positioner is deployed can be envisioned. In the preferred embodiment, the telescoping rod positioner is located at the leading end of a connecting rod placement device. This device consists of a substantially elongated cannula that is designed to be passed through the working cannula which is used to primarily place the pedicle screws themselves. The telescoping rod positioner is found at the leading end of this cannula. Also found within this cannula, in the preferred embodiment, is a flexible core which is secured at its leading end to the innermost cylinder of the telescoping rod positioner. This flexible core can be constructed of almost any elastomeric substance known or acceptable to the art, including polyethylene, polyester, any flexible plastic, polysulfone, or any other such substance. The trailing end of the flexible core is available for manipulation by the surgeon, either directly or via a connecting rod which, when depressed, advances the flexible core. The resultant effect is that the flexible core extends from its trailing end at a point in the shaft of the cannula to its leading end which is reversibly coupled to the innermost cylinder in the telescoping rod positioner, with the flexible core presumably bending, at least to some degree, within the cradle of the trailing end of the screw. This would be found at the leading end of the cannula.

Another mechanism by which the telescoping rod positioner is deployed would be through either pneumatic or hydraulically-driven actuator mechanism. In such an embodiment, there is a delivery channel extending from the trailing end to the leading end. This channel ultimately terminates in a chamber that is located within the expandable component, whether the telescoping or accordion mechanism is being utilized. This will thereby fill the chamber and hence is the actuator which will propel the expandable mechanism into its fully extended position, allowing the leadingmost end to reversibly couple with the leading end of the rod.

Of equal importance to extending the expandable rod positioner is the actuator which compels the expandable element to retract back to its primary position, which is the action that actually positions the rod into proper position in the cradles. In the pneumatic or hydraulic mechanism, several mechanisms can be envisioned which would accomplish this.

In the preferred embodiment, suction is applied to the trailing end of the delivery channel, causing the fluid (air or liquid) to be drawn out of the chamber at the leading end of the mechanism. This will draw the leading end of the fully expanded element back toward the shaft of the device, and (presuming the leadingmost end has coupled with the rod) as this is drawn back it will carry the rod into the preferred position.

An alternative embodiment provides one or more cable or cable-like components to the mechanism. Such a system would be actuated by a mechanism at the trailing end of the device. This mechanism would be provided with an actuator that would allow the cables to be retracted from the trailing end of the device (Which is outside of the patient's body and hence can be easily accessed by the surgeon). These cables would not be strong enough to compel the leading end of the expandable element through the soft tissues which lie between the cradles. However, once the expandable element has been fully deployed, and is hence fully extended and coupled with the rod, the cable system would be strong enough to retract the expandable element, carrying with it the rod.

Yet another method of utilizing the expandable element to position the rod would be to deploy the expandable element, couple with the leading end of the rod, and then for the surgeon to simply manually retract the entire device.

Still other methods for expansion and contraction of either of the expandable elements can be envisioned. One such mechanism is a magneto-electric drive, or magnetically-driven actuator. In such an embodiment, potential magnetic elements can be placed in strategic locations along the expandable elements. When deployment is desired, an electric current is passed into these potential magnets, causing them to become magnetically active, When expansion is sought, the configuration of the electric components is such that like pole are assigned to a scheme such that the like poles are in close proximity to each other, so when the current is applied the poles are compelled to retract away from each other. This, then, then drives the expandable rod positioner. When it was felt appropriate by the surgeon to retract the rod positioner, a different sequence of poles is activated and the device will ultimately retract.

The mechanisms by which the leading end of the telescoping rod positioner reversibly couples with the leading end of the connecting rod may include a jaw-like mechanism, as disclosed above. Alternatively, a configuration may be envisioned in which the leading end of the telescoping rod positioner is provided with a slit-like configuration into which the leading end of the connecting rod can be reversibly fitted, and released therefrom after acceptable positioning of the rod has been achieved. Other alternative embodiments can be envisioned by those skilled in the art.

The preferred embodiment for the coupling of any of the embodiments of the connecting rod placement device has already been disclosed. A number of alternative mechanisms exist by which the leading end of the expandable rod positioner can reversibly couple with the leading end of the engagement platform. In the preferred embodiment, the engagement platform has been provided with an aperture of any geometric configuration. A pair of complementary arms can be a component of the leading end of the expandable rod positioner. When this has been expanded into position, a pair of opposing arms can be deployed from the leading edge end of the expandable rod positioner, these opposing arms capturing the leading end of the elongated member. The expandable rod positioner is then returned to its primary, non-deployed position, and in returning to that position carries the elongated member with it, the leading end of the elongated member now being positioned to couple with the trailing end of the second screw.

In yet another alternative embodiment of the mechanism by which the rod is coupled with the cradles at the trailing ends of the screws, the leading and trailing ends of the rod are provided with a cam which is rotatable around an axis that is also incorporated into [these] terminal portions of the rod. Said axis is oriented in a plane approximately parallel to the long axis of the bone fastening screws, which would dictate that said cams would rotate in a plane orthogonal to this long axis, and within the cradles found at the trailing end of the screws. Furthermore, the axis is positioned approximately half-way along the length of the cam, such that the plane of rotation of the cam would therefore dictate that when the cam was rotated approximately one-quarter turn (90 degrees), two ends of the ends of each cam would be disposed from coupling with the sides of the cradle. As the ends of the cam are brought against the walls of the cradles, one or more crenellations are provided to the aspects of the walls of the cradles which interface with the ends of the cams. This configuration provides a ratcheting mechanism for securing or “locking” the coupling of the cams with the walls of the cradles. Obviously, in such a configuration, there would be no need for the cam-shaft-disc configuration disclosed above.

Another alternative embodiment that would accomplish the goal of positioning the connecting rod in place would be constructing a rod positioning device constructed of a memory-retaining substance such as Nitinol. In a scenario such as that, the leading end of such a device would be developed to be “pre-bent” to the degree necessary but could be straightened out for passage through the working channel, only to re-assume the configuration provided as a baseline. The leading end of this device is then provided with a mechanism for reversibly coupling with the leading end of the rod. This then allows for the target concept to be achieved.

There are also alternative embodiments of the mechanism by which the rod is secured to the cradle-screw complex. One such embodiment would involve the placement of a disc/socket configuration, with the disc achieving reversible coupling with the engagement platform.

Once the elongated member has been brought into position such that the leading end of the elongated member is positioned to be coupled with the trailing end of the secondary screw, a number of mechanisms can be invoked to achieve a relatively irreversible coupling. In the preferred embodiment, as the leading end of the elongated member is positioned within the cradle found at the trailing end of the screw, a securing cap is passed through the insertion cannula. The securing cap is a rounded discoid-like element which is flat on one side. On the other side, the securing cap has been provided with an extension that rises orthogonal to the discoid base of the securing cap. This extension is designed specifically to pass through the aperture on horizontal plate. There are two other extensions, these again being orthogonal to the discoid base and configured so that as the securing cap is compressed against the leading end of the elongated member and the trailing end of the screw, these other two extensions capture the lateral aspects of the horizontal plate and in a “pressure fit” mechanism, force the leading end of the elongated member to be secured against the trailing end of the screw.

In another alternative embodiment of the invention, the leading end of the rod is provided with the engagement platform as provided and disclosed above, while the trailing end of the rod is expanded by a series of concentric expansions creating a series of corrugations, with the smallest being the closest to the central portion of the rod while the largest of these expansions is found in the trailing most end of the rod. These are specifically designed to match a series of irregularities within the archway of the cradle at the trailing end of the secondary screw. When the rod is pulled through, the corrugations match the irregularities of the wall of the cradle, and the trailing end of the rod is secured into position by a “pressure fit” mechanism.

Another alternative embodiment engages a hydraulic mechanism. In this embodiment, the engagement platform of the rod is displaced against the capture surface of the cradle. Within the cradle is the cam-shaft-disc mechanism disclosed above, which in the non-deployed position lies entirely within the cradle. A hydraulic chamber surrounds the cam-shaft-disc complex. As the capture surface is compressed, the hydraulic mechanism is compressed, causing the cam to be displaced from the chamber into the aperture in the engagement platform, thus being positioned to be rotated and locked.

Yet another device that is herein disclosed shall be known hereinafter as the screw inserter. This is an elongated device which shall consist of a leading end, a shaft, and a trailing end.

The leading end is the critical component and is unique insofar that it is specifically configured to capture the cradle/trailing end of the screw complex and stabilize this complex during insertion. The challenges in accomplishing these goals can be appreciated by quickly reviewing the screw and cradle as disclosed above. It can be recalled that the trailing end of the screw is seated within a chamber provided to the leading end of the cradle. This configuration allows for free rotation of the cradle about the trailing end of the screw. Additionally, the cam-shaft-disc complex is coupled with the cradle, such that an insertion device would have to account for this complex as well.

In order to meet these challenges, the leading end of the insertion device is provided with an expanded configuration which is somewhat oblong or eccentric in the dimension orthogonal to the long axis of the shaft of the insertion device. This is specifically configured so that this leading end can be inserted into the trailingmost end of the cradle, and rotated approximately 90° in order to reversibly couple or “lock” the leading end of the insertion device against the medial and lateral walls of the cradle. Through this action the cradle is stabilized. An additional feature of the leading end of the screw insertion device is that there is a central area in the leadingmost end which is configured to mate with the cam of the cradle. When the insertion device is rotated to lock the cradle, the cam-shaft-disc complex is displaced towards the trailingmost end of the screw, relative to the base of the cradle. It can be recalled from the description of the cradle above that such displacement of the leading end of the disc results in reversibly coupling the leading end of this complex against the trailing end of the shall of the screw. Thus, the screw is stabilized, will no longer rotate, and is prepared for insertion.

### BRIEF DESCRIPTION OF THE DRAWINGS

The above and other objects, features, and advantages will become more readily apparent from the detailed description of the embodiments accompanied by the following drawings, in which:

FIG. 1A—Lateral elevational view of the pedicle trocar.

FIG. 1B—Lateral view of the pedicle trocar with cannula being passed over trocar.

FIG. 1C—Lateral view of the pedicle trocars in place within the L5 and Si pedicles; cannulae are being passed into position.

FIG. 2A—Frontal view of bone fastening screw and cradle; exploded.

FIG. 2B—Elevational view of bone fastening screw and cradle; articulated.

FIG. 2C—Elevational view of bone fastening screw and cradle; exploded.

FIG. 3—Elevational view of preferred embodiment of rod, demonstrating engagement platforms.

FIG. 4A—Frontal view of frame of cradle.

FIG. 4B—Frontal view of trailing end of shaft of bone fastening screw and cradle, with cam in unengaged position.

FIG. 4C—Frontal view of trailing end of shaft of bone fastening screw and cradle, with cam being rotated into engaged position.

FIG. 4D—Frontal view of trailing end of bone fastening screw and cradle, with cam locked into fully engaged position.

FIG. 4E—Frontal view of trailing end of bone fastening screw within cradle, demonstrating range of movement of the cradle.

FIG. 4F—Frontal view of trailing end of bone fastening screw within cradle, coupled with leading end of screw insertion device.

FIG. 5—Lateral view of initial position of pedicle trocars with cannulae as they are being passed into the pedicles.

FIG. 6—Cannulae resting against the entry point to the pedicles, lateral view. Trocars are being removed.

FIG. 7—Lateral view of target motion segment with cannulae in place and bone fastening screws being advanced.

FIG. 8—Lateral view of target motion segment with cannulae in place after implantation of screws within the pedicles.

FIG. 9A—Lateral view of telescoping rod positioner in place within the cannula associated with the primary screw and rod within the cannula associated with the secondary screw.

FIG. 9B—Frontal view of telescoping rod positioner in position at the trailing end of the cradle.

FIG. 10A—Lateral view of telescoping rod positioner being deployed.

FIG. 10B—Lateral view of telescoping rod positioner after coupling with leading end of connecting rod and retracting to position the rod in place within the trailing ends of the screw-cradle complexes.

FIG. 11—Top view of rod articulating with cam actuators prior to engagement.

FIG. 12—Top view of rod and cam actuators after engagement.

FIG. 13—Frontal view of the cam locking device engaging the cams after implantation of the construct.

FIG. 14A—Frontal view of screw insertion device.

FIG. 14B—Top view of the leading end of the screw insertion device.

FIG. 15A—Lateral view of the accordion actuator of the expandable rod positioning device.

FIG. 15B—Frontal view of the accordion actuator in position within the trailing end of the cradle.

FIG. 15C—Lateral view of accordion actuator after coupling with leading end of connecting rod; actuator is retracting to position the connecting rod in acceptable position within the trailing ends of the screw-cradle complexes.

FIG. 16—Lateral view of memory substance such as Nitinol being introduced through the cannula of the primary screw and capturing the engagement platforms of the connecting rod.

FIG. 17A—Lateral view of device for rotating and locking the cam in place.

FIG. 17B—Enlarged elevational view of leading end of device for rotating and locking the cam in place.

FIG. 18—Lateral view of connecting rod to be used in two-level construct.

FIG. 19—Two-level construct being implanted

FIG. 20—Lateral view of two-level embodiment construct in place.

FIG. 21—Alternative embodiment in which the rods are provided with a rotatable cam which engages the trailing end of the cradle; lateral view.

### DETAILED DESCRIPTION OF THE DRAWINGS

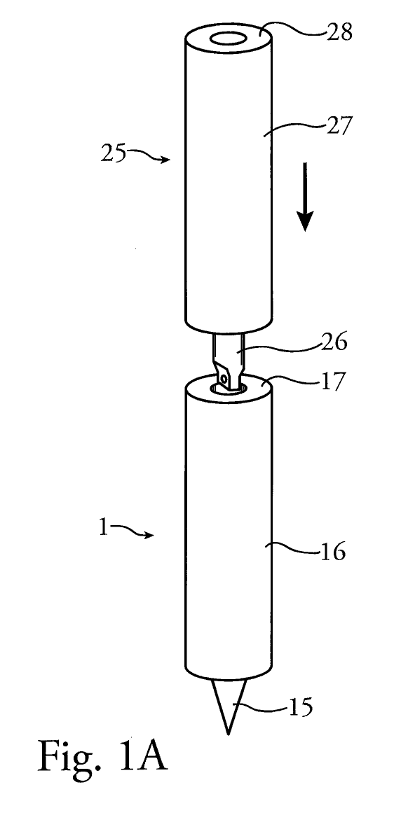

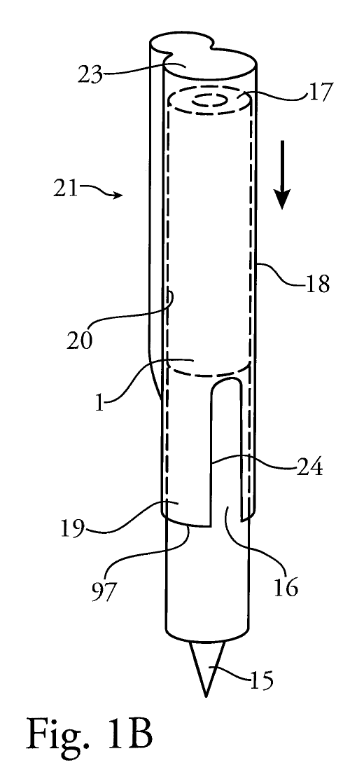

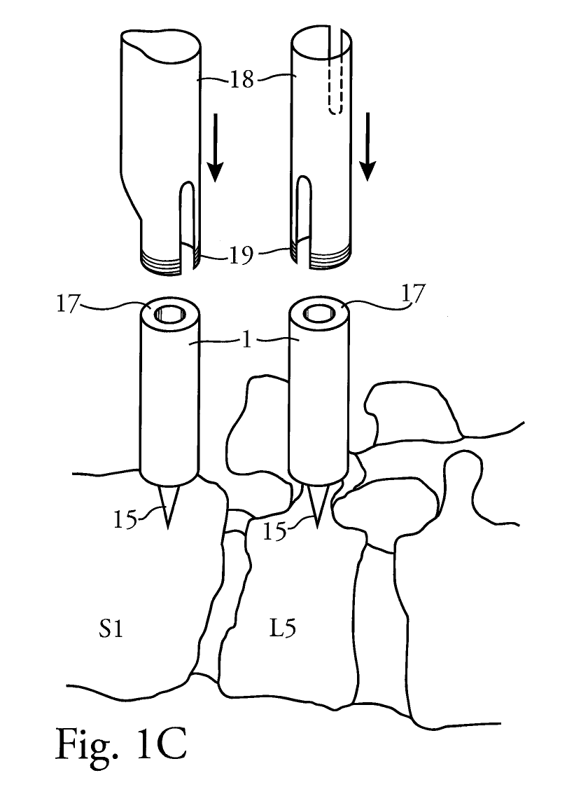

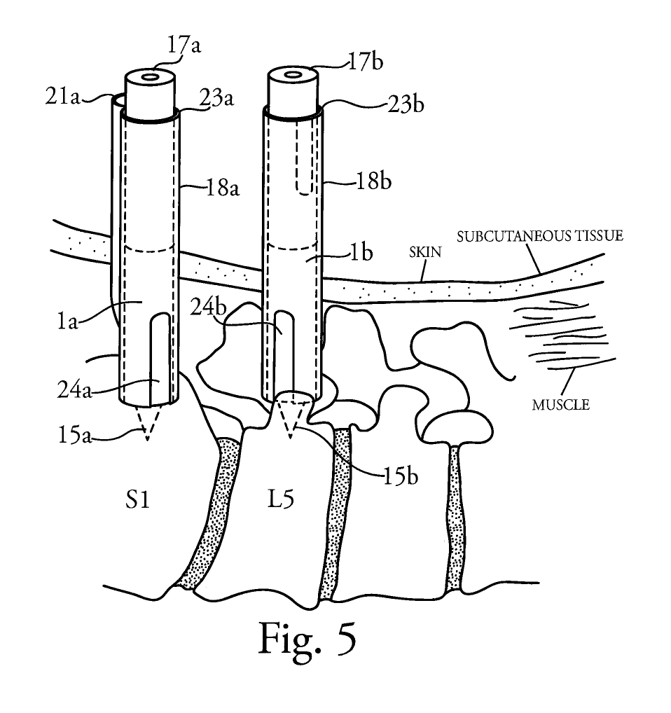

Referring now to the drawings in which like reference numerals identify similar or identical elements throughout the several views, and in particular to FIG. 1A, the pedicle trocar 1 is seen in the lateral elevational view. This is the first device to be used in the implantation of the invention, as demonstrated in FIG. 5. The leading end 15 of pedicle trocar 1 is noted to be tapered, and is composed of a radiopaque substance such as stainless steel, or any other extremely hard metal which can pierce the cortical surface of the entry point to the pedicle. The taper accommodates easier insertion, allowing the device to be insinuated through the soft tissues with greater facility. The shaft 16 gives the necessary length to the pedicle trocar 1; the trailing end 17 of pedicle trocar 1 is widened to approximately the diameter of the working cannula 18 (not shown) which will be inserted in by passing over pedicle trocar 1, as discussed in FIG. 1B. At the trailing end 17 of pedicle trocar 1 is noted a handle 25 which is provided with a leading end 26, a shaft 27, and a trailing end 28. The leading end 26 can be reversibly coupled to the trailing end 17 of pedicle trocar 1. The trailing end 28 of handle 25 can be provided with a variety of embodiments (not shown) by which the surgeon can manipulate pedicle trocar 1 (assuming coupling of the handle 26 to the pedicle trocar 1). Once the pedicle trocar 1 is positioned, handle 25 is uncoupled from pedicle trocar 1 so that the cannula 18 can be then passed into position; again this will be demonstrated in multiple other Figures.

FIG. 1B—The isolated working cannula 18 can be seen passing over the isolated pedicle trocar 1 in this lateral view, demonstrating the fashion by which the cannula 18 is inserted. The arrow demonstrates the direction that the cannula 18 is disposed therethrough. One can also appreciate the difference in diameter between the slender leading end 15 of pedicle trocar 1 and the diameter of cannula 18. Other features of cannula 18 can also be seen, including the leading end 19, the shaft 20, and the trailing end 23. Also seen at the leading end 19 is an example of the stabilizing pointed extensions 97 which reversibly secure the cannula to the bone. Although one such extension is seen in this image, it is presumed that multiple extensions will be found on each cannula. In addition, an extension 21 of cannula 18 can be seen; this extension is designed to accommodate any of the embodiments of the expandable rod positioner in the instance whereupon the cannula 18 is being used in conjunction with the primary screw 4(a) (not shown), as will be demonstrated in several images below. In the setting whereupon the cannula 18 is being used in conjunction with the secondary screw 4(b) (not shown), this extension is utilized to accommodate the connecting rod 35 (not shown) and its placement device 36 (not shown); again, this will be demonstrated in a number of images below. Moreover, implicit in this isolated image is the value of limiting the amount of radiopaque materials in the complex. One can easily realize in these isolated structures, interpretation of the intraoperative images could be most challenging because of excessive metallic artifacts. Also appreciated in this image are the slotted apertures 24 in the working cannula 18 which create a channel for the passage of the expandable rod positioner and the connecting rod 35 (neither shown), as will be demonstrated in numerous images below.

FIG. 1C—In this functional depiction, pedicle trocars 1 have been passed into the pedicles at L5 and Si, which are labeled. The leading ends 15 of the pedicle trocars 1 have been inserted into the entry points of the target pedicles. The cannulae 18 are about to be disposed there through the pedicle trocars 1; the leading ends 19 of the cannulae 18 are slightly larger in diameter than the trailing ends 17 of the pedicle trocars 1; it can be recalled that the trailing ends 17 are the widest parts of the trocars, so once this area is negotiated the cannulae 18 will easily pass over the remainder of the pedicle trocars 1.

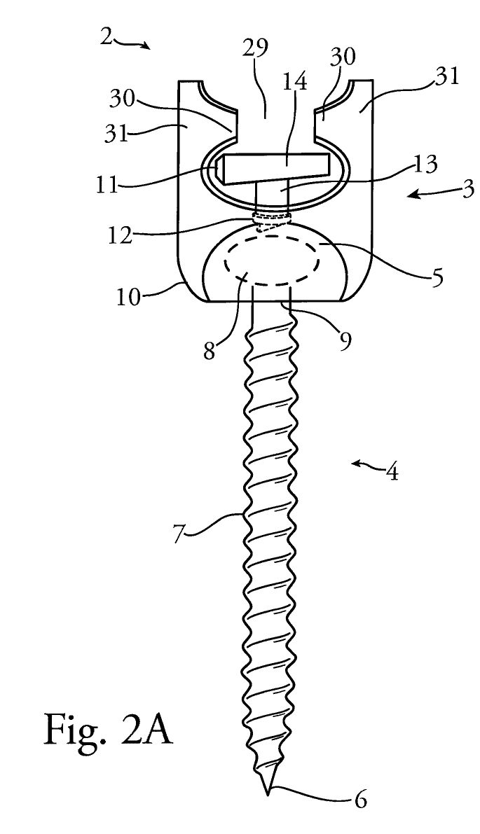

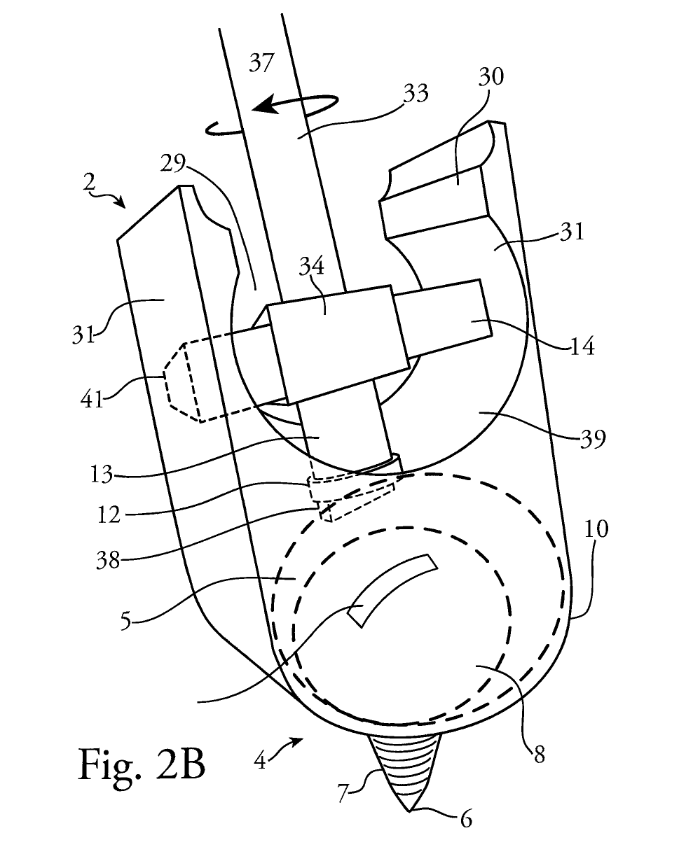

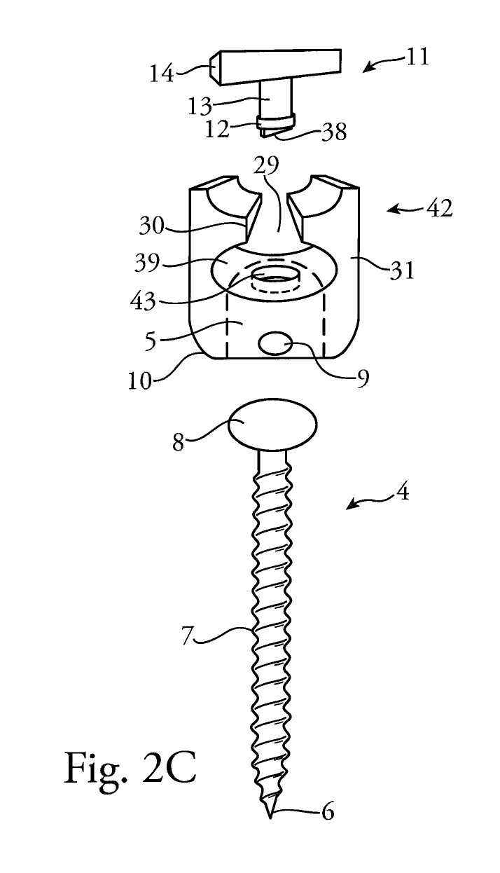

In FIG. 2A, the embodiment of the screw and cradle complex 2 can begin to be appreciated. The cradle 3 is articulating with the trailing end 8 of screw 4, and given the irreversible nature of this coupling or articulation, the cradle becomes the functional trailing end of the screw and cradle complex 2. Also seen in this view is the shaft 7 of screw 4. The trailing end of this shaft 7 passes through an aperture 9 (not well appreciated in this view) in the leading end 10 of the cradle 3. As the result of this configuration, the trailing end 8 of screw 4 is then housed within a chamber 5, which is provided within the leading end 10 of the cradle 3. Because the [expanded] trailing end 8 of screw 4 is substantially larger than the aperture 9 through which the shaft 7 of screw 4 passes, an irreversible coupling between screw 4 and the cradle 3 is created. Also seen in this image is the cam-shaft-disc complex 11, which will ultimately serve to couple with and lock the connecting rod 35 (not shown) in place. This complex 11 is, in the preferred embodiment, unitized, and is comprised of the disc 12, which is found at the leading end of the complex 11 and which will reversibly couple with the trailing end 8 of screw 4 within the chamber 5 at the leading end 10 of the cradle 3. The role of this coupling will be discussed and demonstrated in greater detail in multiple images below. The shaft 13 of this complex 11 translates the rotation of the cam 14 to the disc 12. Also appreciated in this view is the central channel 29 of the cradle 3, which accommodates the connecting rod 35 (not shown), as well as protuberances 30 of the inner walls 31 of the cradle 3. These protuberances 30, as will be discussed and demonstrated in multiple images below, will become critical components in locking the connecting rod 35 (not shown) in place.

These relationships can be also appreciated in FIG. 2B, in which an elevational perspective view demonstrates the screw-cradle complex 2. Again seen is the leading end 6 and shaft 7 of screw 4, which are positioned further from the viewer in this perspective. The trailing end 8 of screw 4 can be seen within the chamber 5 which has been provided to the leading end 10 of the cradle 3. The aperture 9 through which the shaft 7 of screw 4 passes cannot be seen. A slotted depression 32 is provided to the trailing end 8 of screw 4, into which the leading end 38 of the disc 12 may reversibly couple. This slotted depression 32 is just one of any number of conceivable embodiments by which the leading end 38 of the disc 12 may reversibly couple with the trailing end 8 of screw 4; one can envision a circular depression, a hexagonal depression, or any other embodiment; such alternative embodiments would be associated with appropriate changes in the leading end 38 of the disc 12. Any embodiment which allows for this coupling is within the spirit and scope of the invention.

Further demonstrated in this figure is the cam 14, which has reversibly coupled with the leading end 34 of the device 33 which is used to rotate or deploy the cam 14. The cam can be seen “locking” against the inner walls 31 of the cradle 3. The protuberances 30 of the inner walls 31 serve to maintain the cam in the desired position, and cause the cam to be displaced slightly towards the base of the cradle 39. This will, in turn, cause the leading end 38 of the disc 12 to be displaced towards and couple with the slotted depression 32 in the trailing end 8 of screw 4, resulting in coupling of the leading end 38 with the slotted depression 32. This will, in turn, stabilize the entire construct and prevent the cradles 3 from any further rotation around the trailing ends 8 of screws 4. In addition, crenellations 41 on the inner walls 31 of the cradle 3 interface with the cams 14, causing the cam 14 to be “locked” in place. These structures are further exemplified by the elevated exploded view of the screw-cradle complex in FIG. 2C, in which the cam-shaft-disc complex 11 can be seen, as well as the frame 42 of the cradle 3, and screw 4. The implicit coupling of the cam-shaft-disc complex 11 with the frame 42 of the cradle 3 can be appreciated; an aperture 43 at the base 39 of the central channel 29, through which the shaft 13 of the cam-shaft-disc complex 11 passes. Owing to the fact that the cam 14 and the disc 12 are substantially larger than the diameter of this aperture 43, this configuration creates an irreversible coupling between the frame 42 of the cradle 3 and the cam-shaft-disc complex 11. Other aspects of the frame 42 of the cradle can also be seen, including the inner walls 31 with the protuberances 30 which jut into the central channel 29. These protuberances 30 serve to capture the cam 14 when it is in the fully-deployed position. Also seen are the chamber 5 provided to the leading end 10 of the cradle 3, and the aperture 9 which the shaft 7 of screw 4 is disposed therethrough; one can again appreciate the coupling of the trailing end 8 of screw 4 with the leading end 10 of the cradle 3. It can be seen in this view that the trailing end 8 of screw 4 is substantially larger than the diameter of the aperture 9 at the leading end 10 of the cradle 3. This creates an irreversible coupling between the cradle 3 and the trailing end 8 of screw 4. An additional feature of this image is the chamber 5 which is provided to the leading end 10 of the frame 42 of the cradle 3. This chamber 5 houses the trailing end 8 of screw 4. By studying the aperture 9 at the leading end 10 of the frame 42 of the cradle 3 and the aperture 43 provided to the base 39 of the central channel 29 of the frame 42 of the cradle 3, it can be recognized that these two apertures lead into the same chamber 5. Through this configuration, the leading end 38 of the disc 12 can couple with the trailing end 8 of screw 4, specifically by coupling with the slotted depression 32 provided to the trailing end 8 of screw 4.

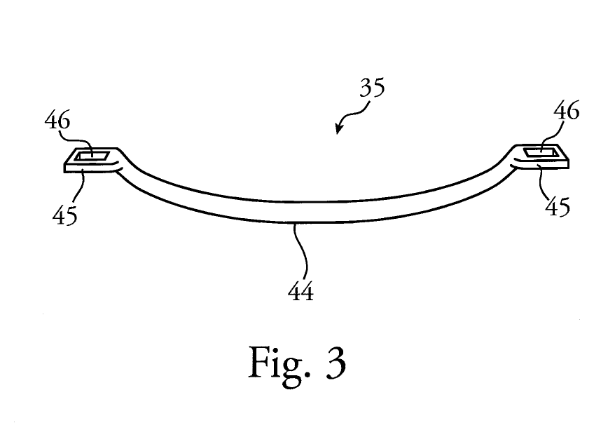

FIG. 3 demonstrates a lateral view of the connecting rod 35. One can appreciate the lordotic curvature which has been provided to the central expanse 44 of the rod 35. At each end can be seen the flattened, horizontal expanses of the engagement platforms 45. These are oriented orthogonal to the long axis of the screws 4 (not shown in this Figure). The central apertures 46 located within the engagement platforms 45 are designed to couple with the cams 14 of the cradle 3 (neither structure shown), as previously discussed.

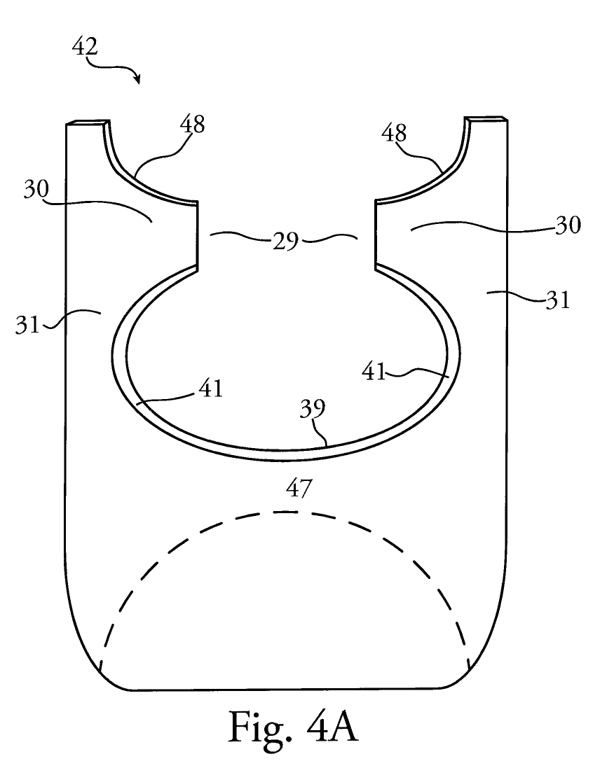

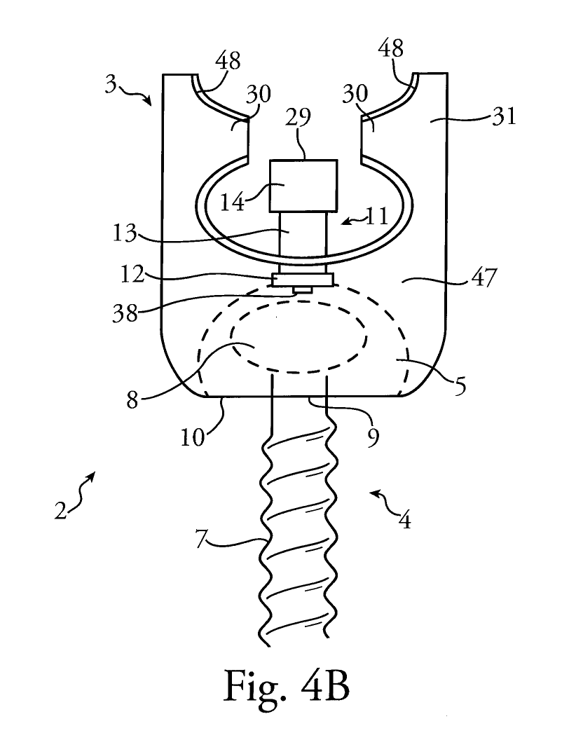

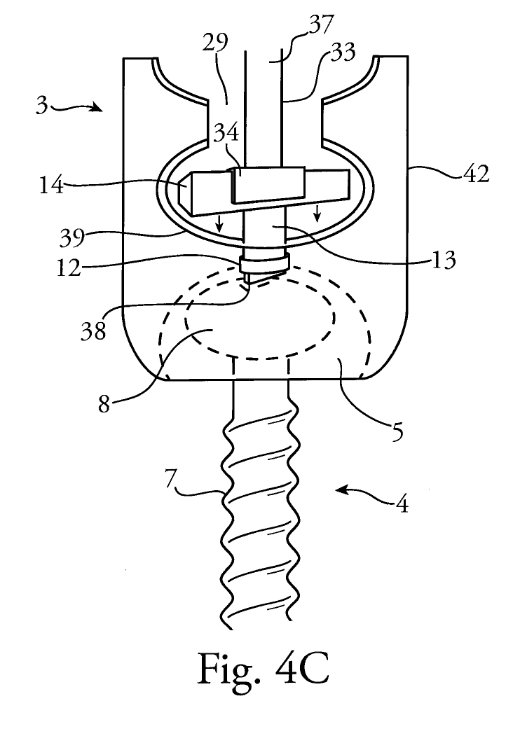

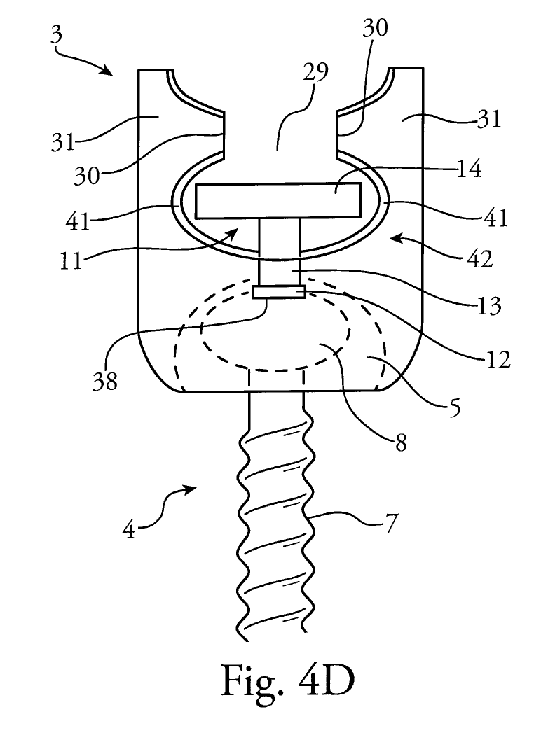

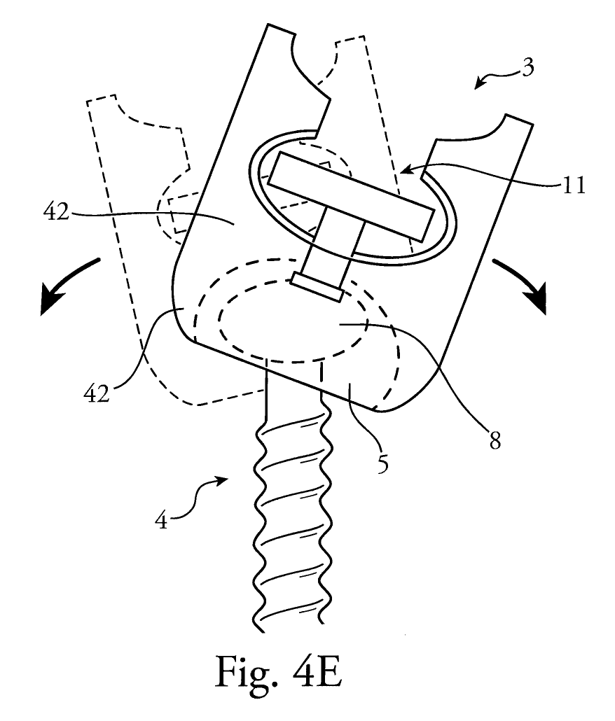

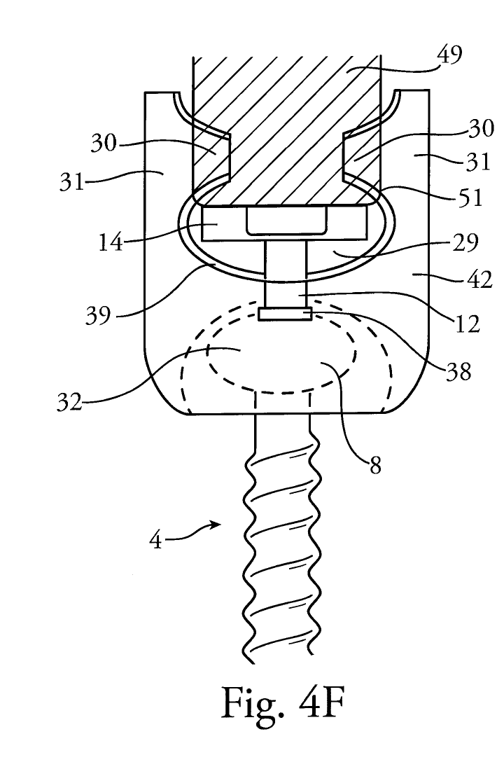

FIG. 4A demonstrates the frame 42 of the cradle 3 in the frontal view. The substantially “H” shape can be appreciated, as the result of the bridging element 47 which separates the central channel 29 superiorly from the chamber 5 inferiorly, this chamber having been provided to the leading end 10 of the frame 42. The walls 31 can be seen projecting superiorly from the base. These projections are more substantial at the areas closest to the base, and are widest at the point furthest from the base. The rod channel 48 is represented by the widest portion of the channel found in the superior aspect of the central channel 29. The walls are provided with symmetric protuberances 30 which divide the central channel 29 into the superior channel or rod channel 48 above and the base or capture surface 39 below. The specific design of the walls 31 of the allows for free rotation of the cam 14 (not shown) when there is no engagement platform 45 (also not shown) being encased within the channel 29, as the cam 14 can be fully displaced against base 39 of the cradle 3; however, when the engagement platform 45 is present, then the cam 14 (neither 14 or 45 shown) cannot be displaced completely against the base 39 of the central channel 29 but can only be displaced to the limits as dictated by the thickness of the engagement platform 45. In that case, when the cam 14 is rotated it will be locked against the walls 31 of the channel 29 by crenellations 41, which are not well appreciated in this image but have been provided to the inner surfaces of the walls 31.

In FIG. 4B, a frontal view of the screw-cradle complex 2 demonstrates the shaft 7 of screw 4 which is continuous with the [expanded] trailing end 8. The shaft 7 can be appreciated as it is transmitted through a [presumed] aperture 9 at the leading end 10 of the cradle 3, although the aperture 9 is not actually seen in this projection, A chamber 5 in the leading end 10 of the cradle 3 has been provided with an expanded configuration recapitulating the configuration of the trailing end 8 of screw 4. This configuration results in a moment of rotation of the cradle 3 around the trailing end 8 of screw 4. This allows the cradle 3 to align with the cradle 3 of screw 4 that has been inserted into the adjacent pedicle (not shown), particularly in the setting whereby the pedicles are not pursuing the same angle of junction with the vertebral body—which is a common occurrence. Also seen is the horizontal or bridging element 47 of the frame 42 of the cradle 3, along with the walls 31 forming the central channel 29. The superior portion of the central channel 29 is wider and represents the rod channel 48. This is separated from the base 39 of the central channel 29 by the protuberances 30 extending from the walls 31. Protruding into the central channel 29 is the cam 14 and shaft 13 of the cam-shaft-disc complex 11. The disc component 12 of this complex is seen inferior to the bridging element 47, which forms the base 39 of the central channel 29. The leading end of the disc 38 is seen within the chamber 5 of the leading end 10 of the frame 42 of the cradle 3. The leading end of the disc 38 can be seen in close approximation to the trailing end 8 of screw 4; it is important to note, however, that these structures are not coupled when the cam-shaft-disc complex 11 is in the non-deployed position, as depicted in this image. The irreversible coupling of the cam-shaft-disc complex 11 to the frame 42 of the cradle 3, as seen in FIG. 4C, allows for the rotation of the cam-shaft-disc complex 11, which is a central element of the invention and provides one of the principal objects of the invention. As seen in this image, the cam-shaft-disc complex 11 is being rotated by the cam rotation device 33, the leading end 34 of which is engaging the cam 14. The shaft 37 of this device 33 can be seen passing through the central channel 29 to accomplish this. This device 33 engages the cam 14 and displaces the cam-shaft-disc complex 11 towards the base 39 of the central channel 29. This action also displaces the leading end 38 of the disc 12 towards the trailing end 8 of screw 4. Arrows demonstrate the anticipated direction of this displacement.

The cam-shaft-disc complex 11 has been rotated into the fully deployed position in FIG. 4D. The cam-shaft-disc complex 11 has been rotated within the central channel 29 into the fully deployed position such that the cam 14 is now interfacing with the crenellations 41 of the walls 31 of the cradle 3. When an engagement platform (not shown) has been disposed therethrough, the cam 14 cannot be fully displaced against the capture surface, and when the cam is rotated it will be locked against the walls of the channel. This is accomplished by the crenellations 41 (not seen in this perspective) found on the inner surface of the walls 31.

Importantly, as demonstrated in this image, the leading end 38 of the disc 12 has been displaced such that it is now coupled with the trailing end 8 of screw 4, preventing further rotation of the cradle around the trailing end 8 of screw 4. As previously stated, this eliminates the moment of rotation of the cradle 3 around the trailing end 8 of screw 4.

The advantage of this moment of rotation provided to the cradles accounts for variations in the angles that the pedicles emerge from the vertebral bodies. This can vary from vertebra to vertebra, and is particularly true in the degenerated lumbar spine. This can present a great deal of difficulty when attempting to pass a connecting rod between the trailing ends of screws implanted within these vertebrae, but the moment of rotation demonstrated in FIG. 4E can help to reconcile these varied angles. Here it can be appreciated that because of the geometric configuration of the trailing end 8 of screw 4 within the chamber 5 found at the leading end 10 of the frame 42 of the cradle 3, specifically because the chamber 5 is of the same configuration but slightly larger than the trailing end 8 of screw 4, the cradle 3 can vary in its alignment with the long axis of screw 4. This is demonstrated in this image, whereupon the primary position of the cradle is drawn in solid lines while stippled lines represent a possible variation in the alignment of the cradle 3 with the screw 4. Arrows demonstrate the anticipated degrees of freedom.

FIG. 4F—In this frontal view, the leading end of the screw insertion device 49 to insert the screw into the bone is demonstrated. The configuration of the screw insertion device 49 specifically accomplishes this goal of the invention by providing the screw insertion device 49 with an inner component and an outer component (not shown). Resulting from this configuration, the leading end of the inner component 50 reversibly couples with the cam 14 in such a fashion so as to displace the cam-shaft-disc complex 11 towards the base 39 of the central channel 29. This displacement, in turn, will cause the leading end 38 of the disc 12 to engage and reversibly couple with the slotted depression 32 of the trailing end 8 of screw 4. Furthermore, rotation of the inner component 50 of the screw insertion device 49 will result in rotation of the cam-shaft-disc complex 11 which will, via the coupling of the leading end 38 of the disc 12 with the trailing end 8 of screw 4, result in rotation of screw 4. Critically, the leading end 51 of the outer component of the screw insertion device 49 can be seen engaging the trailing end of the cradle 3 including the protuberances 30 of the walls 31 of the frame 42 of the cradle 3. In doing so, the screw insertion device 49 will stabilize the frame of the cradle 42; in this fashion, the screw 4 can be rotated via the mechanism described above without rotation of the cradle. This will result in successfully implanting the screws 4 into the target pedicles.

FIG. 5 is a lateral view of the initial steps involved in implantation of the invention. The target pedicles in this example are L5 and Si, which have been labeled. It is recognized that the working cannulae 18 are going to be positioned at both levels; ultimately, one of these shall be arbitrarily chosen to be the cannula through which the expandable rod positioner (regardless of the specific embodiment thereof) shall be passed. For clarification, the screw with which this cannula is associated shall be known as the primary screw and is assigned the working identification of screw 4(a). The second screw shall be known as the secondary screw and is assigned the working identification of screw 4(b); in keeping suit, the other components used at both levels shall also retain the [lettered] sub-classification/working identification.

Therefore, pedicle trocars I are seen in this lateral projection having been passed through the skin and subcutaneous tissues (demonstrated in portion of figure), with the leading end of the trocars 15 piercing the entry point to the pedicles. The trocars are ideally made of radiolucent material with the leading ends 15 being made of a radiopaque metal. The S I screw shall be nominated as the primary screw in this example and therefore the sub-classification (a) is assigned to the leading end 15(a) of the pedicle trocar 1(a) which is being placed into the Si vertebra. The cannulae 18(a, b) have already been passed over the trocars 1(a, b); these are separate steps but have not been illustrated sequentially. The apertures 24(a, b) in the cannulae 18 (a, b) through which the connecting rod 35 is ultimately passed are seen. The trailing ends of the trocars I7(a, b) and the cannulae 23(a, b) are seen above the skin as the surgeon utilizes these to complete further steps in the operation, Also, the extension 21(a) of the cannula 18(a) is seen on the cannula being utilized at Si.

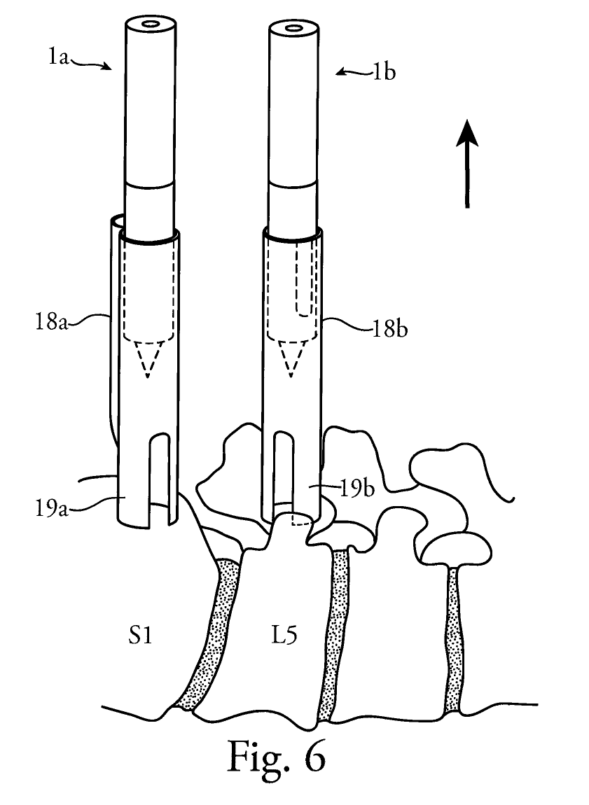

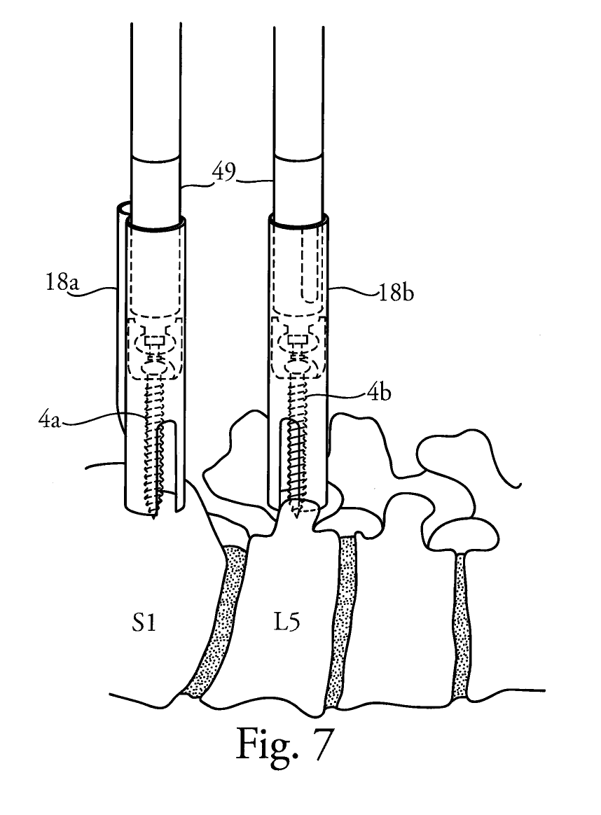

In FIG. 6, the leading ends 19(a, b) of cannulae 18(a, b) are seen resting against the bony entry points to the pedicles of L5 and S 1—again, this could apply to any segment but the example being given here is L5-S1. The plurality of stabilizing pointed extensions 97 (not demonstrated) can then be deployed to anchor the leading ends I.9(a, b) of cannulae 18(a, b) into the bone at the entry points to the pedicles. The pedicle trocars 1(a, b) are being removed, as indicated by the directionality of the arrow. These cannulae 18 (a, b) will then provide working channels through which the remainder of the procedure can be accomplished, as demonstrated in FIG. 7 in which the screws 4(a, b) coupled to the screw insertion devices 49 and can be seen being advanced into these working channels/cannulae 18(a, b). It should be noted that in the embodiment demonstrated herein, the screws 4 are self-drilling and self tapping; in an embodiment in which drilling and tapping are required, these steps would have already been performed in this image.

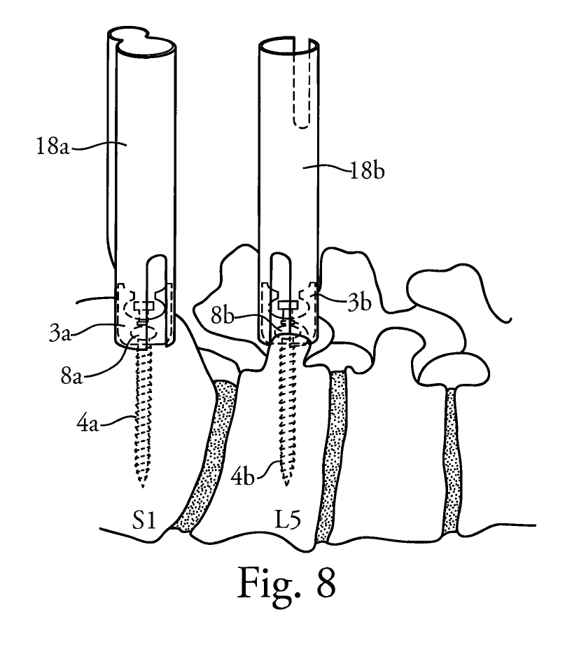

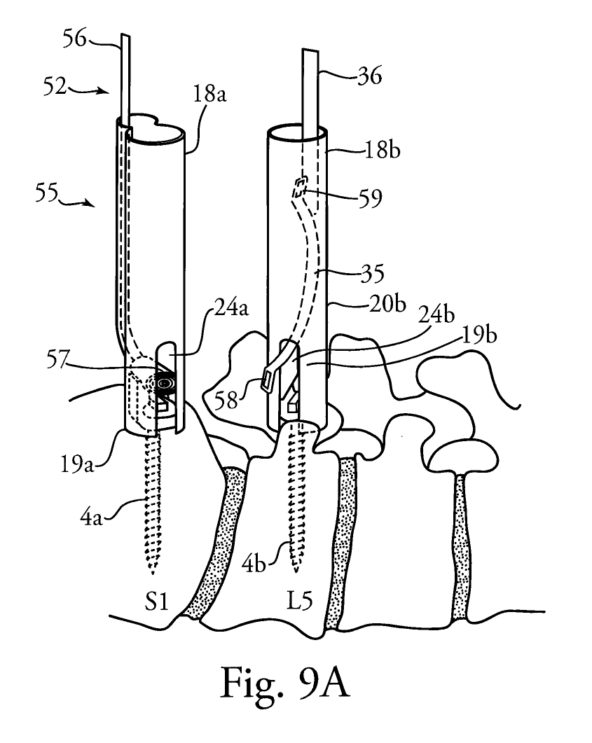

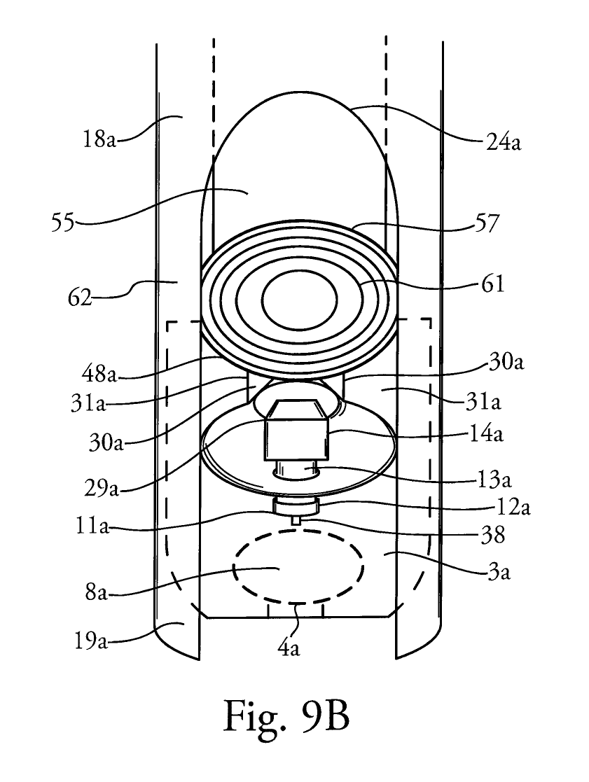

In FIG. 8, the cannulae 18(a, b) are in place and the screws 4(a, b) have been advanced into the bone of the pedicles of L5 and SI as labeled. The cradles 3(a, b) at the trailing ends 8(a, b) of the screws 4(a, b) are now prepared for positioning of the connecting rod (not shown), a process which commences in FIG. 9A with placement of the telescoping embodiment 52 of the expandable rod positioner into cannula 18(a) associated with primary screw 4(a) which has been implanted into Si and as depicted in the lateral view herein. This is placed specifically into extension 21(a) of cannula 18(a). The telescoping rod positioner 52 is comprised of the telescoping element 57 seen at the leadingmost end, and a shaft 53 which is in turn comprised of a leading end 54, a midportion 55, and a trailing end 56. The expandable portion may be deployed by a number of different mechanisms, including a mechanical mechanism (as demonstrated herein) by which a flexible rod 53 is advanced into the interior of the telescoping element 57 causing it to deploy/expand (this action not demonstrated in this image). Also included in the application but not depicted herein is a pneumatic mechanism and/or a hydraulic mechanism which can be utilized to deploy the telescoping element 57. The telescoping element 57 can be seen positioned at aperture 24(a) of cannula 18(a), such that deployment of the telescoping element 57 will cause this to expand toward the aperture 24(b) of the cannula 18(b) associated with the secondary screw 4(b), which has been implanted into L5. In this fashion, the telescoping element 57 will expand toward the leading end 58 of the connecting rod 35, which has been positioned within the lumen of the shaft 20(b) of cannula 18(b) associated with the secondary screw 4(b). The connecting rod 35 has been inserted into the cannula 18(b) with the use of the rod positioner 36, which is reversibly coupled to the trailing end 59 of the connecting rod 35. This can also be appreciated in a frontal view seen in FIG. 9B whereby concentric circles 61 represent the geometric arrangement of the elements of the telescoping rod positioner 52 as they would appear in a frontal perspective. This expandable element 57 of the telescoping rod positioner 52 is within the aperture 24(a) of the leading end 19(a) of the cannula 18(a), which has been passed over and positioned with relationship to the cradle 3(a). In particular, one notes that the expandable element 57 of the telescoping rod positioner 52 is resting in the superior portion 48(a) of the central channel 29(a), as it is actually resting on the dorsal aspects of the protuberances 30(a) of the walls 31(a) of the cradle 3(a). Because of this configuration, the expandable element 57 is seen above the cam 14(a) of the cam-shaft-disc complex 11(a). The trailing end 8(a) of the primary screw 4(a) can also be seen.

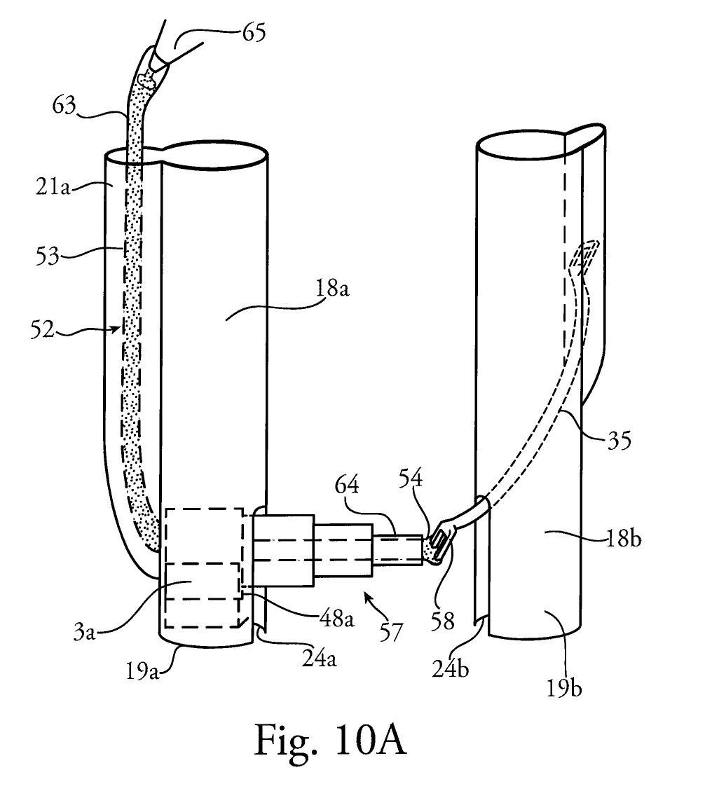

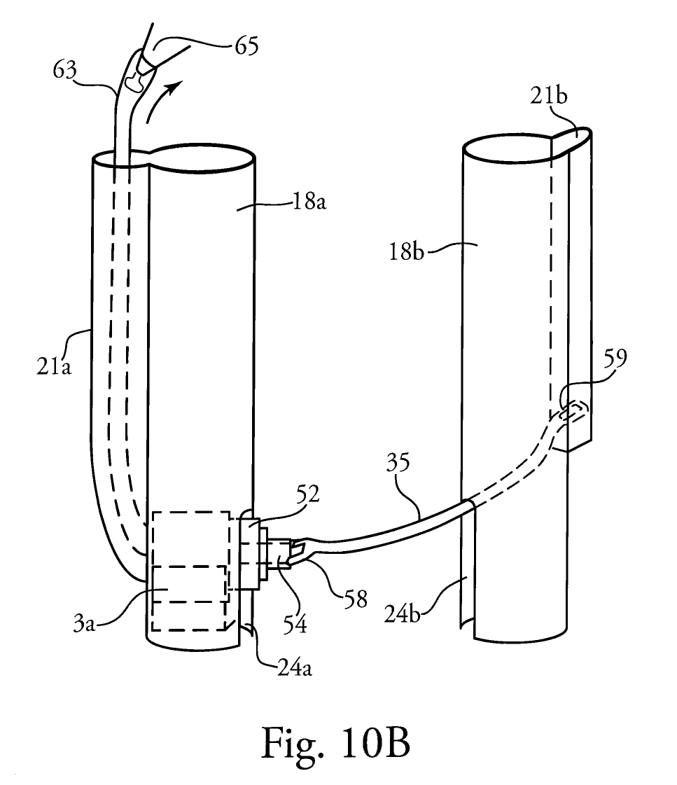

Deployment of the telescoping rod positioner 52 can be accomplished by several mechanisms; in FIG. 10A, the actuator which is demonstrated utilizes a flexible rod 63 which passes through the shaft 53 of telescoping rod positioner 52, this shaft having been positioned in the extension 21(a) of cannula 18(a), and is deployed by a purpose-specific instrument, the leading end 65 of which is seen in this image. The leading end 64 of flexible rod 63 reversibly couples with the interior of expandable element 57, compelling this element to expand through aperture 24(a) at the leading end 19(a) of cannula 18(a) towards the leading end 58 of connecting rod 35 which can be seen protruding through an aperture 24(b) at the leading end 19(b) of cannula 18(b) associated with the secondary screw 4(b) (screws not shown in this image). The leading end 54 of the telescoping rod positioner 52 then couples with the leading end 58 of the connecting rod 35. As has already been demonstrated, the leading end 54 of the telescoping rod positioner 52 is seated within the superior chamber 48(a) of the central channel 29(a) of the cradle 3(a).

The telescoping rod positioner 52 can be compelled to expand by one of several mechanisms, including a pneumatic, hydraulic, or manual actuator. Regardless, once the actuator has been activated, the various elements are displaced with respect to each other, resulting in expansion of the device. Upon expansion, the leading end 54 of the telescoping rod positioner 52 will then couple with the leading end 58 of the connecting rod 35, in preparation for final positioning of the rod 35 in conjunction with coupling the cradle 3 to the rod 35.

Retraction of the telescoping rod positioner 52 back towards cannula 18(a) associated with the primary screw (not shown) is actuated by the direction of the deployment device 65 as indicated by the arrow. As demonstrated in FIG. 10B, this action results in repositioning the connecting rod 35 so that it may easily pass into the cradles 3(a, b) of both screws (not shown in this image). The final positioning of the connecting rod 35 is further augmented by the fact that the telescoping rod positioner 52 will ultimately retract into extension 21(a) of cannula 18(a), so that when the leading end 54 of telescoping rod positioner 52 uncouples with the leading end 58 of connecting rod 35, the leading end 58 of connecting rod 35 is positioned directly over cradle 3(a) in preparation for being displaced into the cradle 3(a) for locking in place by deployment of the cam locking mechanism 33 (not shown in this image).

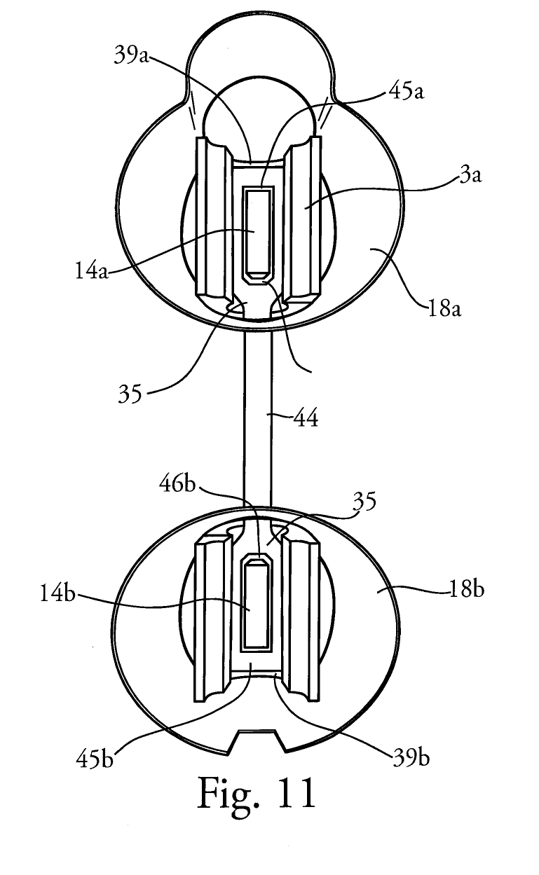

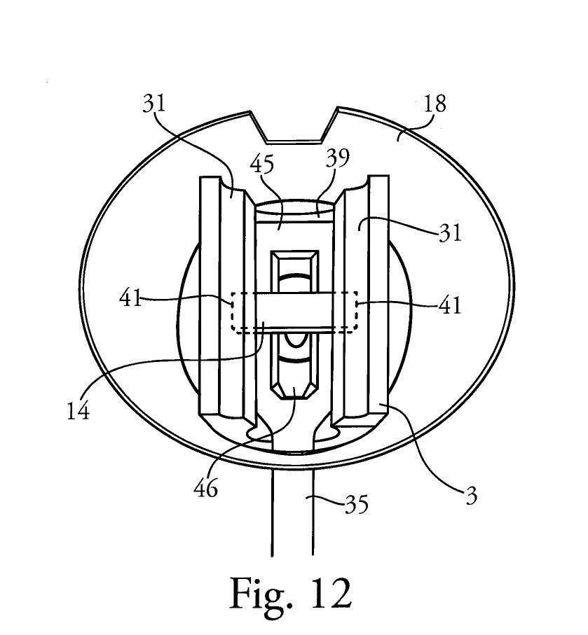

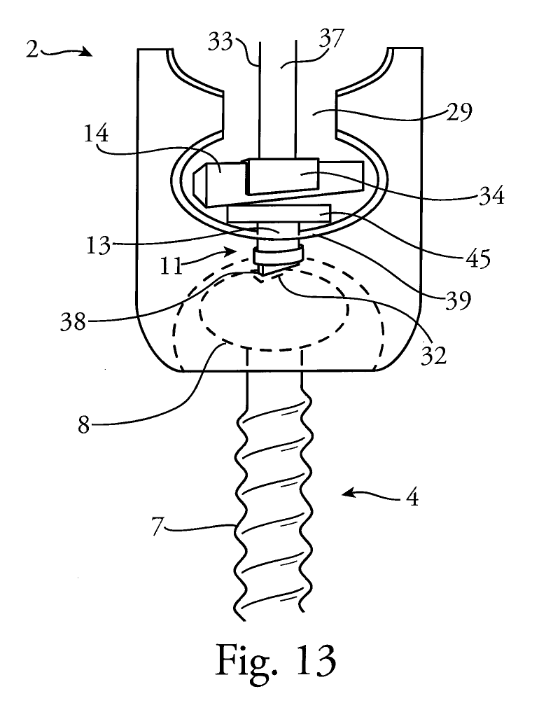

This can be best understood with the “top view” perspective offered in FIGS. 11 and 12. In these images, the viewer's perspective is as viewed through the working channels of the cannulae 18(a, b). It must be recognized that in actual practice, the central expanse 44 of the connecting rod 35 would not be seen as it would be a subsurface structure at this point in the procedure; however, the connecting rod 35 is demonstrated here so that it can be seen for illustrative purposes with the engagement platforms 45(a, b) seated within the cradles 3(a, b). In FIG. 11, the long axes of the cams 14(a, b) are aligned with the greatest diameter of the central apertures 46(a. b), thus allowing the engagement platforms 45(a, b) to be displaced towards the capture surfaces/bases of the central channels 39(a, b) specifically so that deployment can be achieved easily and efficiently. In the second image (FIG. 12), which is demonstrating a single representative complex as viewed through the cannula 18, the “locking” mechanism has been deployed and the screw 4 (not shown in this image) and connecting rod 35 are now in final position. The cam 14 has been rotated 90° so that its lateral edges are interfacing with the crenellations 41 along the inner walls 31 of the cradle. The engagement platform 45 has been displaced against the capture surface 39 of cradle 3. The cam 14 is now at a 90° angle to the long axis of the aperture 46 of the engagement plate 45. This can be further appreciated in FIG. 13 in which the fashion by which the cam secures the rod into the cradle is further demonstrated. In this frontal image of a representative screw-cradle complex 2, the shaft 7 and trailing end 8 of screw 4 are noted, as well as the shaft 37 and leading end 34 of the device 33 which rotates the cam 14, said shaft 37 passing into the central channel 29 of the cradle 3. The leading end 34 of this device 33 can be seen coupling with the cam 14 and rotating the cam 14 into locking position. The engagement platform 45 of the connecting rod 35 (not shown) can be seen resting against the base 39 of the central channel 29. The engagement platform 45 is secured between the cam 14 and the base 39 of the central channel 29. In this fashion, the engagement platform 45 and therefore the connecting rod 35 (not shown) are secured into position within the cradle 3 of the screw-cradle complex 2. It is also noted that the leading end 38 of the cam-shaft-disc complex 11 is displaced against and into the slotted depression 32 by translation of the displacement of the cam 14 through the shaft 13 causing the leading end 38 of the disc 12 to be displaced into the depression 32 of trailing end 8 of screw 4, thereby locking and stabilizing the entire construct.

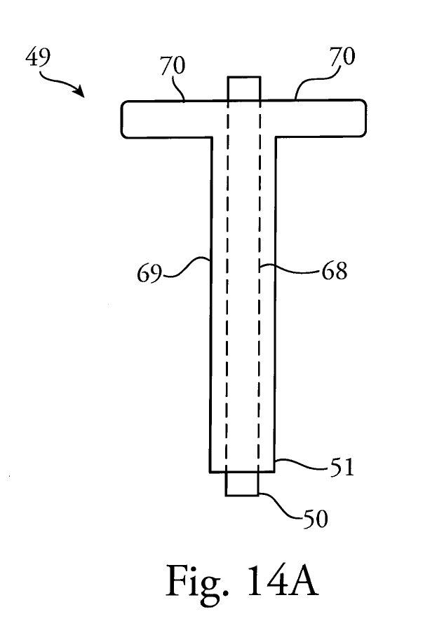

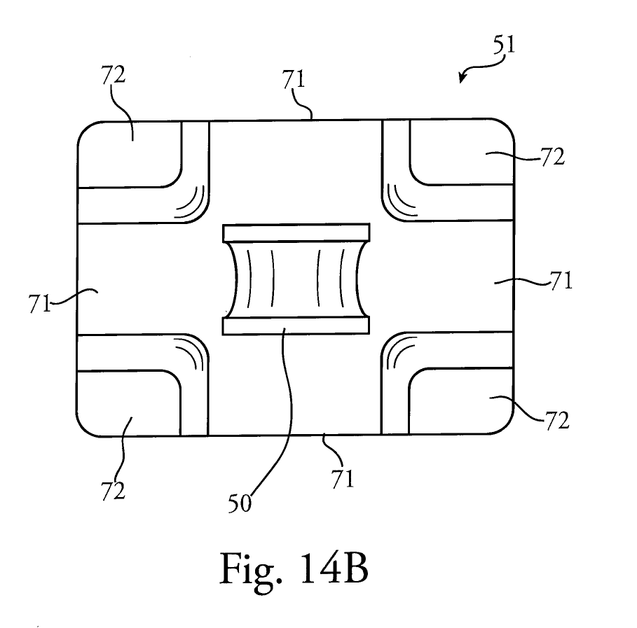

FIG. 14A depicts a lateral view of the device 49 utilized to implant the screw-cradle complex 2 into the bone. This device is comprised of an inner component 68, which, as depicted, is an elongated element which extends from the trailing end 70 of the device 49 to the leading end 51 with the leading end 50 of the inner component 68 protruding through an aperture at the leading end 51 of the outer component 69, and extending beyond the leading end 51 of the outer component 69. This arrangement allows for the inner component 68 to rotate independently; furthermore, the device 49 is configured so that when the leading end of the inner component 68 reversibly couples with the cam 14, the leading end 51 of the outer component 69 will reversibly couple with the cradle 3, stabilizing the cradle 3 and preventing the cradle 3 from moving. The sum result is that the cam 14 and therefore the screw 4 can rotate independently during insertion of the complex 2 into the target pedicle. This is further illustrated in FIG. 14B in which a detailed confrontational view of the leading ends 50 and 51 of the device 49 is seen. The leading end 50 of the inner component is noted to have an interior surface and configuration which will easily mate with the cam 14 and compel the cam 14 to rotate. This rotation in turn is translated to the trailing end 8 of screw 4 (not shown) via the previously described coupling of the cam-shaft-disc complex 11 to the trailing end 8 of screw 4. However, the entire screw-cradle complex 2 (not shown) does not rotate during insertion owing to a unique configuration of the leading end 51 of the outer component 69 which stabilizes the cradle 3 during implantation. This is accomplished via a configuration which provides a series of lofts 72 which in turn create a sequence of depressions 71, ultimately creating a contour which will mate with the protuberances 30 of the cradle 3, thereby stabilizing the cradle 3 during implantation.

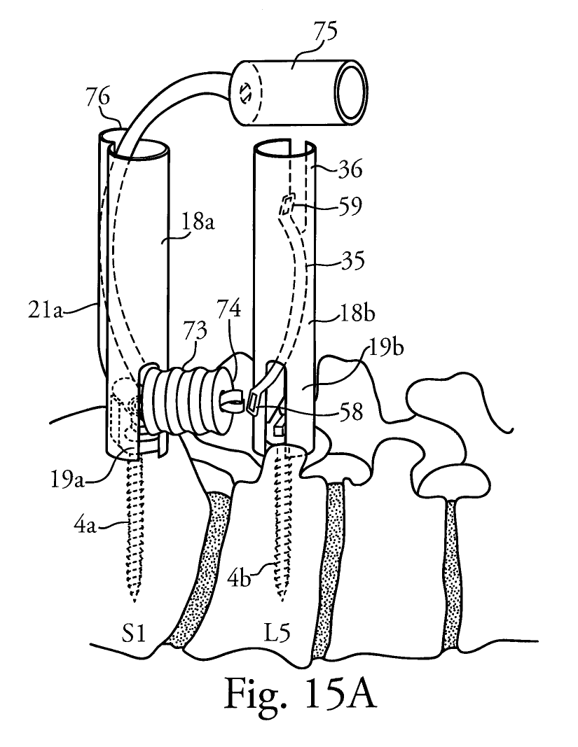

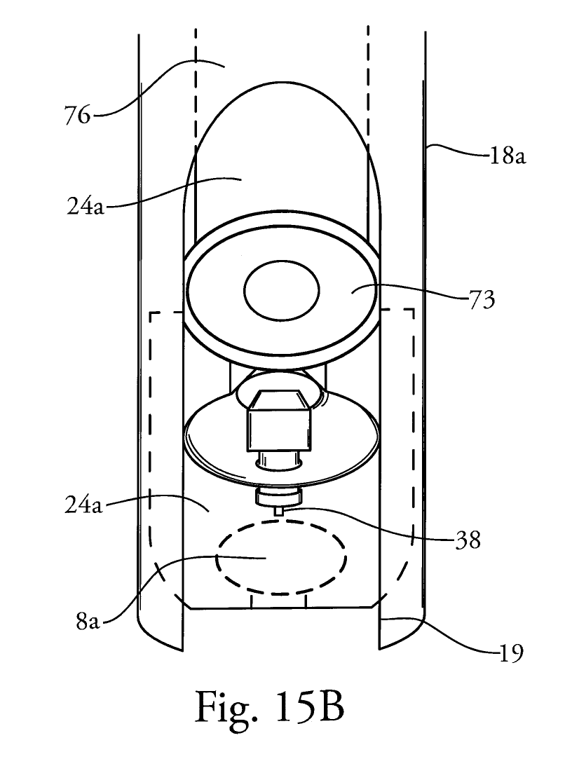

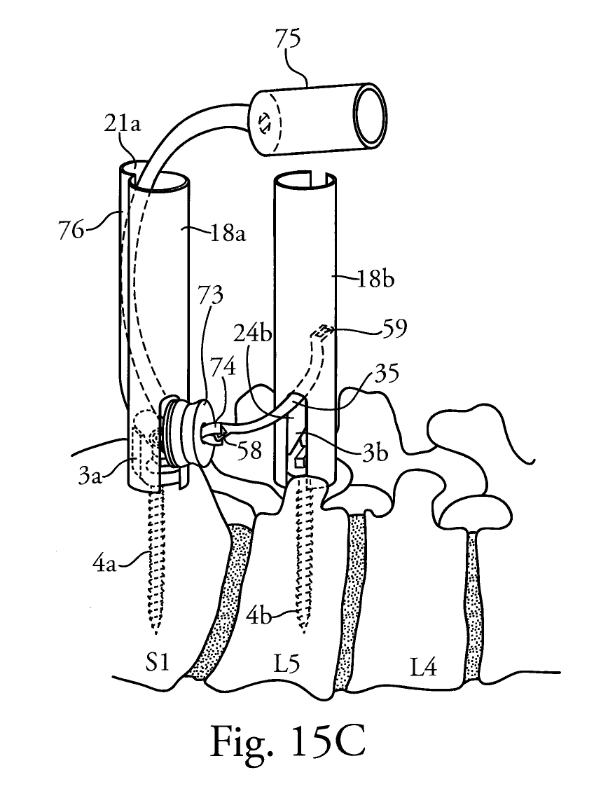

FIG. 15A—In this lateral perspective view, both cannulae 18(a, b) are in plaCe at the trailing ends of screws 4(a, b), and a different embodiment of the expandable rod positioner, in this instance an accordion expandable rod positioner 73 is in position within the leading end 19(a) of cannula 18(a) associated with the primary screw 4(a). The accordion expandable rod positioner 73 has been deployed, has extended through aperture 24(a) of cannula 18(a), and has traversed the span between the two cannulae 18(a, b). The accordion expandable rod positioner 73 has been deployed, in this instance, utilizing a pneumatic actuator 75 which is connected to the accordion expandable rod positioner 73 via a connecting tubing 76. Again, other actuator mechanisms could be utilized, such as hydraulic, mechanical, magneto- electric, and any actuator mechanism which is known or acceptable to the art, and all of these are within the spirit and scope of the invention. The connecting rod 35 can be seen in position within the leading end 19(b) of cannula 18(b) associated with the secondary screw 4(b). The leading end 74 of the accordion expandable rod positioner 73 is provided with a jaw-like mechanism which will capture and reversibly couple with the leading end 58 of the connecting rod 35. It is noted that the connecting tubing 76 has been disposed through the extension 21(a) of the cannula 18(a).

FIG. 15B—The relationship of the leading end of the accordion expandable rod positioner 73 to the leading end 19(a) of cannula 18(a) can be further appreciated in this frontal view, wherein the non-deployed accordion expandable rod positioner 73 seen within the aperture 24(a) found at the leading end 19(a) of cannula 18(a). The involuted accordion expandable rod positioner 73 can be noted to be in an ideal position to expand towards the cannula 18(b) with the leading end 58 of rod 35 contained therein (not shown).

The full effect of the actuator mechanism of the accordion can be seen in FIG. 15C, in which the accordion expandable rod positioner 73 has retracted back towards the cannula 18(a) associated with the primary screw 4(a). Owing to the reversible coupling of the leading end 74 of the accordion expandable rod positioner 73 with the leading end 58 of the connecting rod 35, the connecting rod 35 has now been brought into a position to couple with cradles 3(a, b) of screw-cradle complexes 2(a, b). After the accordion expandable rod positioner 73 has retracted bringing the connecting rod 35 into the leading end 19(a) of cannula 18(a), the accordion expandable rod positioner 73 is then retracted into the extension 21(a) of cannula 18(a), assuring that the leading end 58 of the connecting rod 35 is ideally positioned with respect to the cradle 3(a) of the primary screw 4(a). In an analogous fashion, the trailing end 59 of the connecting rod 35 is positioned to couple with the cradle 3(b) of the secondary screw 4(b).

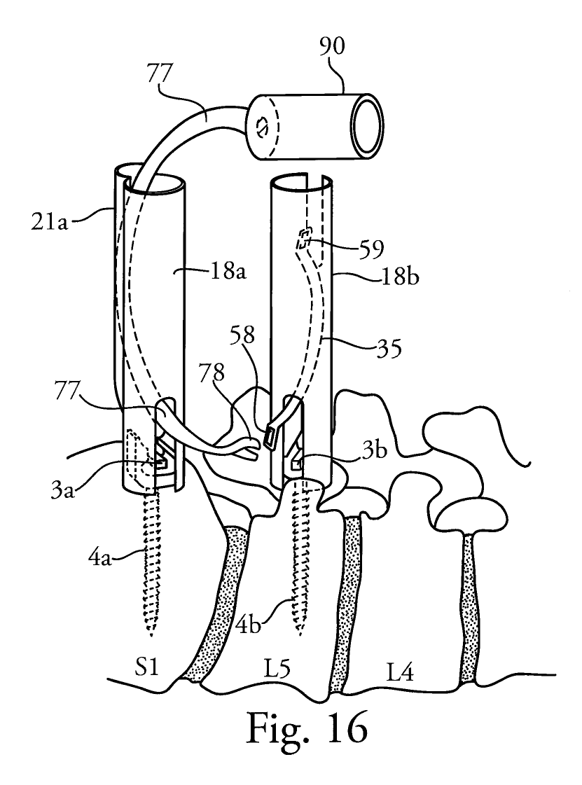

FIG. 16—In this lateral view, cannula 18(a) associated the primary screw 4(a) is being used to introduce a rod positioner 77 composed of a memory-retaining substance such as Nitinol. Such a device is flexible such that it can readily be disposed through extension 21(a) of cannula 18(a) and, once introduced, will reassume its original curvature as it extends towards the cannula 18(b) of the secondary screw 4(b); in doing so, the leading end 78, which has been provided with a jaw-like configuration, couples with the leading end 58 of the connecting rod 35. The Nitinol rod positioner 77 will then be withdrawn, carrying the connecting rod 35 into final position. The procedure is then completed in the same fashion as described for the other embodiments of the rod positioners disclosed above.

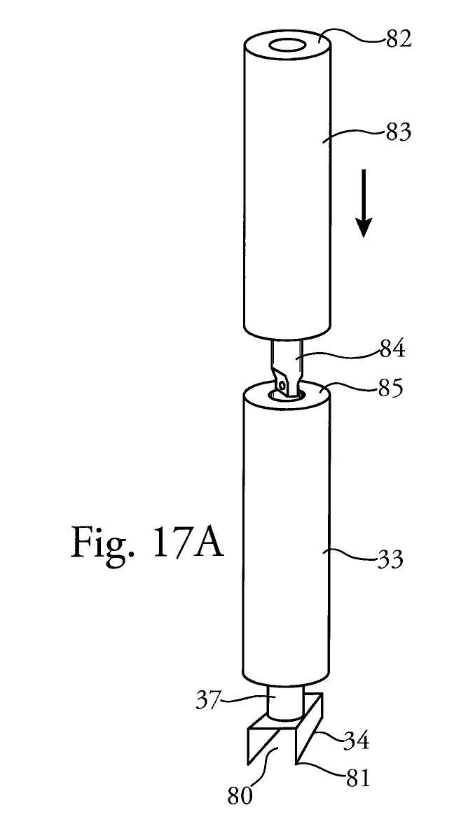

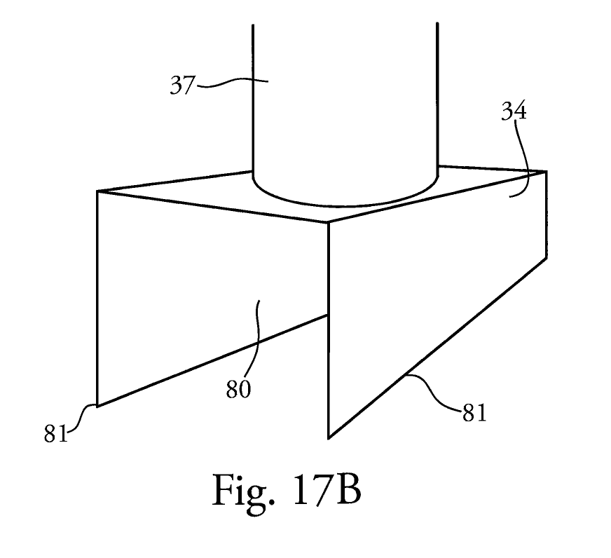

FIG. 17A—Device 33 for rotating and locking the cam in place. A handle 83 is reversibly coupled with the trailing end 85 of the device 33. A channel 80 into which the cam 14 (not shown) is transiently accommodated is noted at the leading end 34 of the device 33. The depth of this channel 80 is sufficient such that the cam and shaft (not shown) can be contained entirely within the leading end 34 and the leadingmost edge 81 of the channel 80 will interface with the engagement platform (not shown); slight downward manual pressure (direction indicated by arrow) on the trailing end 82 of the handle 83 will cause the cam 14 and the engagement platform 45 to be displaced flush against the capture surface 39 of the cradle 3. These structures and this action are not illustrated in this image. The leading end 34 can be understood in greater detail in FIG. 17B, in which an enlarged view of the leading end 34 can be seen. The shaft 37 of the device 33 is seen extending into the leading end 34, which is essentially an “inverted U-shape” and thus is provided with a channel 80 into which the cam 14 and shaft 13 can be reversibly coupled. The leading end 34 is configured specifically to accommodate the cam 14. The leadingmost edge 81 will interface with the engagement platform 45 of the leading end 58 of the connecting rod 35.

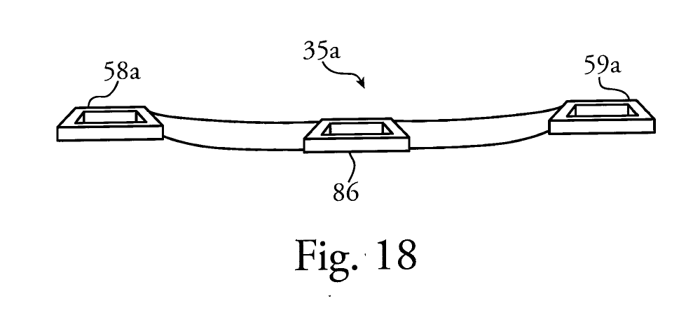

FIG. 18—An embodiment of a connecting rod 35(a) designed for a two-level construct. In this embodiment, there is seen a leading end 58(a) which would be initially introduced into the cannula associated with the secondary screw (neither structure shown). There is also an intermediate engagement platform 86 which would couple with the cradle associated with the intermediate screw (not shown). Ultimately, the leading end 58(a) would couple with the cradle 3 of the primary screw 4(a) and the trialing end 59(a) would couple with the trialing end 8(b) of the secondary screw 4(b).

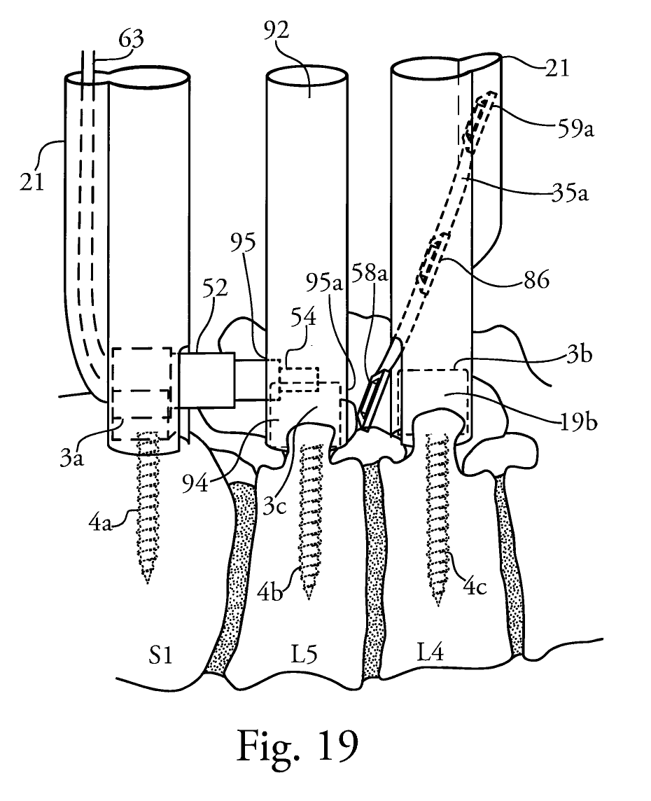

In FIG. 19—A two-level construct can be seen during implantation. Three cannulae are in place, namely cannula 18(a) the leading end 19(a) of which is coupled to the cradle 3(a) of the primary screw 4(a), as well as the leading end 19(b) of cannula 18(b) which is coupled with the cradle 3(b) of the secondary screw 4(b); the leading end 94 an intermediate cannula 92 is coupled to the cradle 3(c) of an intermediate screw 4(c). It is anticipated that in the preferred method a telescoping rod positioner 52 is provided which can span as many levels as necessary. In doing so, the leading end 54 of the rod positioner will extend from its position in the cannula 18(a) extending through the apertures 95, 95 A at the leading end 94 of the intermediate cannula 92 and ultimately extend to capture the leading end 58 A of the connecting rod 35 A which has been passed through the secondary cannula 18(b). The telescoping rod positioner 52 is then retracted, and in doing so will position the rod 35 A in such a fashion that it can be coupled to the cradles [3(a, b, and c)] coupled to the pedicle screws 4(a, b, and c). The intermediate engagement platform 86 permits coupling with the cradle 3(c) of the intermediate screw 4(c).

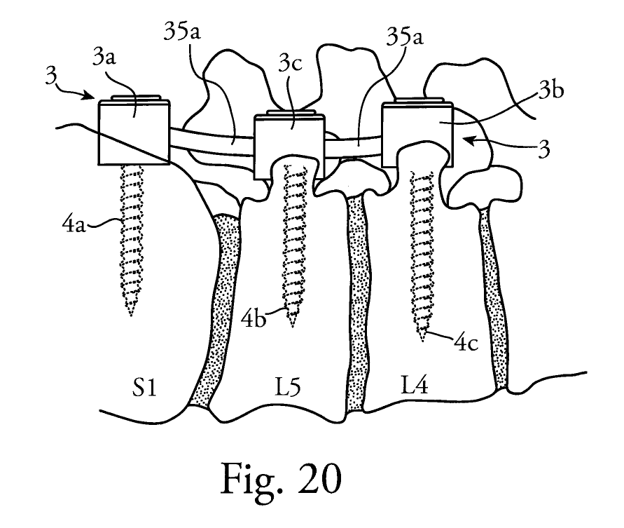

FIG. 20 demonstrates the implanted two-level construct in place from the L4 to the S I vertebrae (levels have been labeled). The screws 3(a, b, and c), of course, have been implanted into the vertebrae through the pedicles, and therefore would not be seen from this perspective but (as in previous images) are schematically included for the edification of the reader. Cradles 3(a, b, and c) are seen at the trailing ends 8(a, b, and c) of the screws 4(a, b, and c). It can be assumed that the connecting rod 35(a) has been coupled with and secured to the cradles in the same fashion as disclosed above for the single-level construct.

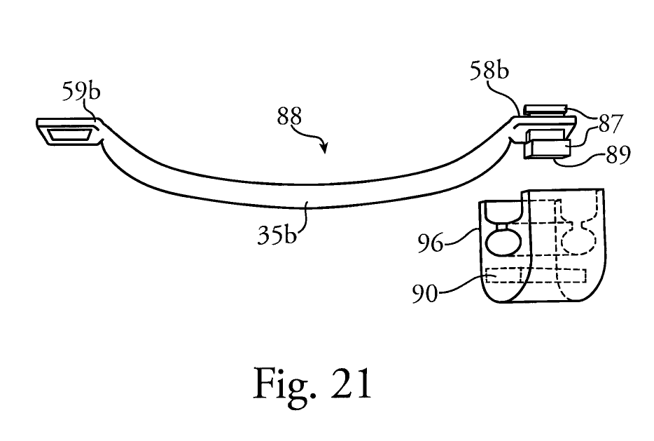

In FIG. 21, an alternative embodiment is demonstrated; specifically, in contrast to the primary embodiment of the invention, in this alternative embodiment 35(b) the ends of the rods 58(b) and 59(b) are provided with rotatable cam-shaft-disc complexes 87. This is in contrast to the preferred embodiment, in which the trailing ends 58 and 59 of the connecting rod 35, are provided with the engagement platforms 45 and the cam mechanism 11 is part of the cradle 3. In this alternative embodiment, there is a modification of the connecting rods 35(b) and cradles 96 such that the ends 58(b) and 59(b) of the connecting rod 35(b) have been provided with a cam-shaft-disc complex 87 and, in turn, this cam-shaft-disc complex 87 will engage the alternative embodiment of the cradles 96, which have been provided with apertures/chambers 90 this action being similar in overall function to that of the primary embodiment. The cradles 96 have been specifically reconfigured to accommodate the cams 89 once inserted. The cams 89 will be passed into apertures 90 in the cradles 96, and a mechanism similar to that in the primary embodiment will allow for this action to lock the trailing end of the screw proper, the cradle, and rod together so as to eliminate any moment of rotation of the cradle around the screw head. This mechanism is not shown in this illustration.

### ALTERNATIVE EMBODIMENTS

Several alternative embodiments can be conceived of in terms of the method of securing the trailing end of the screws to the connecting rod. In one embodiment, the cradle as well as the connecting rod is identical to that disclosed above, except that the cam is not present. Rather, in this embodiment, a locking cap is utilized.

This locking cap is a flattened, discoid-shaped device which is substantially flattened on side. On the other side, the locking cap is provided with several features that are monolithic with and arise from the discoid base. The most prominent of these is a central strut. This is designed in such a fashion that the leading end of the strut is attenuated, with the strut becoming more robust at the trailing end near the base. This arrangement will allow for this central strut to be inserted into the central aperture of the horizontal expanse of the connecting rod. The locking cap is inserted into the channel of the cradle, with the narrower leading end being that portion that of the strut that is inserted into the aperture, with the wider base being secured against the walls of the cradle that create the channel. The strut is coupled with the cradle using a “pressure-fit” mechanism for being secured into position.

In addition, there are two additional extensions arising from the surface of the discoid base and configured such that these press against the external surface of the walls of the cradle. When this securing cap is coupled with the cradle, this combination of factors provides for an extremely secure coupling. It is noted that in this embodiment of securing the rod to the cradle, the outside walls of the cradle are configured such that there is a slight downward sloping, with a corresponding configuration of the extensions of the locking cap.

However, it is essential to be able to remove such locking caps and indeed the entire construct at times, and to this end the following is disclosed.

The outermost extensions of the locking cap are provided with tabs, perforations, or any other irregularities by which the extensions can be easily grasped by a purpose-specific grasping device which can then lift the securing cap off of its coupling with the cradle.

In yet another alternative embodiment of the cradle and mechanism for locking the rod within the cradle, it is noted that the cradle is provided with somewhat thickened walls of the channel. These walls are not monolithic, as in the other embodiments disclosed herein, but rather these walls are separate components that can be slidably repositioned. Moreover, these separate components can be slidably repositioned and then held in place with the use of a ratcheting mechanism that has been provided to this embodiment. In the ideal embodiment, once the horizontal expanse of the rod is in place, these components are slidably repositioned to capture the terminal portions of the rod and in that way secure the rod to the cradle. As in the case of the locking cap, removal is essential and can be accomplished by slidably repositioning the components of the cradle and removing the rod.

Another alternative embodiment of the cam-disc element provides for this element to be somewhat larger in size, with a central channel which would then permit passage of the leading end of an insertion device such as a screwdriver which would then pass through this channel so that it may be inserted into an appropriate place on the trailingmost end of the hemisphere or other embodiment of the trailingmost end of the screw.

### Embodiments Designed for Use in Multiple Levels

An important additional consideration in spine surgery involves cases in which degenerative disease or other pathologic processes exist at multiple levels—i.e. more than a single motion segment. Often, particularly in the setting of advanced degenerative disease, treatment may involve placement of pedicle screws and connecting rods at 2, 3 or [rarely] more levels. It is even more common to utilize multiple levels in the treatment of trauma, and neoplasia of the spine.

Of course, when considering this concept, it is recognized by the inventor that when implanting a construct utilizing “minimally-invasive” procedures, a pragmatic reality suggests that it would be unlikely that a construct would involve more than three motion segments; that notwithstanding, it must also be recalled that this system of devices and methods for use may include so-called “open” spinal procedures that may involve a greater number of motion segments. Therefore, such a construct may contain any conceivable number of motion segment levels. Hence, the rods utilized in this setting could be provided with any conceivable number of engagement platforms would be included within the spirit and scope of this invention.

Therefore, embodiments must be provided that allow the devices provided for and disclosed in this invention to be implanted at multiple levels. In order to accomplish this object of the invention, several embodiments may be envisioned.

In the preferred embodiment addressing this issue, a variation of the connecting rods, as well as a variation of the expandable rod placer is provided. In this iteration, a single rod is disclosed which is configured in such a way that it can be coupled with the cradles of multiple screws placed in multiple adjacent levels of the spine. This is accomplished by disclosing a rod which is provided with multiple engagement platforms, the precise number of which are contingent upon the number of levels/target pedicles which have been proposed for the construct. If the construct is proposed to span and unite in fusion two motion segments (two levels) such as in the example of an L4-S I fusion, then a total of three pedicle screws (per side) would be placed.

Hence, the rod utilized in this embodiment would be provided with three engagement platforms (L4, L5 and Si). Conversely, should a fusion extending from L1 to L5 were to be proposed, then a total of 5 screws on each side would be implanted; therefore, the rod would be provided with 5 engagement platforms. It is therefore recognized that variations of this rod would be manufactured which would contain 3 or more engagement platforms, depending on the number necessary to achieve the desired endpoint.

Another factor to be considered in such a construct is the morphology of each individual patient's spine. Given the fact that individuals vary substantially in size from one person to another, it is easily recognized that a difference in the height of patients would predict a difference in inter-pedicular distance (Although this difference may not be proportionately as great). This, then, predicts a difference in the length of the rod necessary.

Therefore, a critical consideration in conjunction with the number of motion segments being included in the construct is the proposed craniocaudal expanse/dimension of the construct. This dimension can vary—sometimes substantially—and must be accounted for in the overall length of the rod to be utilized.

When considering constructs involving a single level, this is generally not a major factor, regardless of the size of the patient; however, when larger (multi-level) constructs are entertained, the craniocaudal dimension of the construct may vary substantially with the size of the individual patient.

It would, however, be ideal to limit the number of sizes of rods that are ultimately produced. This would result in optimizing important considerations such as inventory management and inventory velocity, and more directly reduce undue confusion and misunderstandings in the operating theatre.

Therefore, the rods provided for this embodiment would vary in length. The engagement platforms would be provided at reasonable intervals along the length of the rod. In order to insure accessibility of the cradles to the engagement platforms, it is envisioned that the mid-position engagement platforms are more extensive in the craniocaudal direction than the standard engagement platforms. As in the case of implantation at a single motion segment described above, the length of the rod being utilized for this multi-level construct can be predicted by the relationship of the cranial most insertion cannula to the caudal most insertion cannula.

There is also a variation of the telescoping rod positioner necessary to position the rod. In this variation, a rod positioner is provided which can extend two or more motion segments, reversibly couple with the leading end of the rod, and then retract and in doing so position the rod such that the engagement platforms are positioned to be secured into the cradles.

In the preferred embodiment, this is accomplished by providing a device which is almost identical to the device utilized in the primary preferred embodiment utilized in the placement of a single level construct, that device having been completely described above. In the case where a multiple level construct is being proposed, the leading end of the rod positioning device is provided with an “accordion-like” mechanism which is specifically designed to extend over several motion segments.

This mechanism can be deployed by a variety of mechanisms. It can be brought into the “extended” position, as well as positioning the rod by retracting through a pneumatic/hydraulic actuator mechanism. This was completely described above, and would function identically whether in the setting of a single or multiple-level fusion.

Speaking to those considerations, an alternative method for creating a multi-level construct is disclosed. A principal component of this embodiment is a variation of the cradle. This variation is specifically designed to be utilized in the intervening or “middle” screw of a proposed construct. For example, in a construct involving implantation of pedicle screws into L4, L5, and S 1, the screw bearing this variation would be found at L5. Extending this concept, if a construct extended from L3, L4, L5 and S1, then the L4 and L5 screws would be provided with this variation of the cradle. This variation shall be hereinafter known as the connecting cradle.

In this variation, the cradle is provided with a stem which is substantially longer than that which is found in the standard cradle. This allows for one rod to be passed from the cradle associated with the pedicle screw superior to the connecting cradle, while a second screw is passed from the cradle associated with the screw implanted into the pedicle inferior to the connecting cradle. These two rods are then connected at the connecting cradle, causing the two rods two act effectively as a single unit.

It can be envisioned that utilizing this technique, multiple intermediate screws that have been provided with a connecting cradle can be utilized to create a multi-level construct. For example, a construct connecting Li-L2-L3-L4 and L5 would utilize screws that have been provided with connecting cradles at L2, L3 and L4, with standard cradles being provided to the screws at L1 and L5. Multiple rods are then implanted, with one rod traversing from L1 to L2. A second rod then extends from L2 to L3, and the leading end of the first rod as well as the trailing end of the second rod are both coupled with the cradle of the L2 screw, being secured into place by the cam of the L2 cradle which secures both engagement platforms. Similarly, a third rod extends from L3 to L4 and a fourth rod extends from L4 to L5. The L3 and L4 cradles are connecting cradles and secure engagement platforms from the superior and inferior rods at each of these levels.

Another issue regarding the invention involves the natural curve, or lordosis, of the spine. This can be seen in lateral radiographs. It is anticipated that the vast majority of patients who would benefit from this technology would suffer disease of the lowermost lumbar spine and lumbosacral junction. In this instance, the necessary lordosis is well-known to all involved. However, in certain instances other areas of the spine may require such intervention, and rods may be provided with varying degrees of lordosis. All such variations would be included within the spirit and scope of this invention.

Once the target motion segments have been defined and the pedicle screws have been implanted, implantation of the rod into the cradles at the trailing ends of the screws must be completed. In this embodiment, as stated above, this is accomplished through the use of a variation of the telescopic rod positioner.

In this variation, the expandable rod positioner is passed through the insertion cannula associated with at least one of the central screws. This rod positioner is provided with not one but two expandable elements. One of these elements is positioned so as to expand in a cranial direction; the other expandable element is positioned so as to expand in a caudal direction. The construct associated with this variation of the expandable rod positioner, there is not one rod but rather a separate rod for every motion segment in the construct. In order to achieve this, the rods are coupled with the cradles of the intermediate screws as the result of a unique variation of the cradle. In this variation, the length of the shaft of the cam-shaft-disc complex is long enough to capture the engagement platforms of both rods. Furthermore, the cradles have been modified to accommodate both platforms.

In all of the disclosures above, the embodiments described are representations of preferred and alternative embodiments of this invention in all its components and perturbations. It is recognized that those skilled in the art may devise and develop unique variations of these embodiments, but such variations remain within the spirit and scope of this invention.

What is claimed:

1. A spinal stabilizing system comprising: at least one connecting rod-like elongated member, a plurality of bone fastening screws that are implantable into the pedicles of the superior and inferior vertebrae comprising at least one target motion segment, wherein said screws being then coupleable to said connecting rod and this system thereby being capable of stabilizing and hence eliminating any between the vertebrae of the target motion segment.

2. The bone fastening screws of the system of claim 1 above wherein the trailing ends of said bone fastening screws is provided with a cradle which couples with said connecting rod-like elongated member.

3. An elongated connecting rod having a modified leading end and trailing end so fashioned so as to engage and be secured within the trailing ends of the cradles which have been coupled to the primary and at least one secondary bone fastening screw.

4. The elongated connecting rod member of claim 3, which is configured to have substantially flattened, horizontal platform or engagement platform which has been provided with a planar axis which is orthogonal to the long axis of the bone fastening screws; furthermore, this flattened horizontal platform is provided with a central aperture which is configured so as to participate in the mechanism by which the screws are secured to the elongated connecting rod element.

5. The cradle of claim 2 which is irreversibly secured to the trailing end of the bone fastening screws wherein said cradle being provided with a central channel which is configured so as to couple with the engagement platform of the elongated connecting rod element.

6. The cradle in claim 2 further comprising a cam, a shaft, and a disc that cooperate with one another to provide fixable connection between said pedicle screw and said connecting rod.

7. The disc element of claim 6 which is irreversibly coupled to the cradle, that cradle in turn being irreversibly coupleable to the trailing end of bone fastening screws with the coupling fashioned and configured in such a way as to provide the cam with a rotatable property.

8. The elongated connecting rod of claim 1 further comprising an aperture which has been provided to the engagement platforms at the leading and trailing ends of the elongated connecting rod.

9. The cam of claim 6 which is oriented in the primary position so that the greatest dimension of the cams are aligned in axis with the greatest dimension of the apertures found in the leading and trailing ends if said connecting rod.

10. A device which can be inserted through the working cannulae in claim comprising a trailing end which can be deployed by a surgeon, a shaft which can be inserted through the cannula(e) and a leading end which can reversibly couple with said cam.

11. The device of claim 10 wherein when deployed, can displace and rotate said cam prior to deployment to a position oriented orthogonal to the deployed position.

12. The Cradle of claim 6 wherein said cradle has an internal wall and said internal wall is provided with one or more crenellations to provide a ratcheting type mechanism to said cam so that said cam can be coupled to the wall of the cradle.

13. An expandable rod positioner comprising a leading end, a shaft, and a trailing end and a means for expanding the leading end of said device, and where said expandable rod positioner is insertable through a cannulae.

14. The expandable rod positioner of claim 13 where said expandable leading end can expand orthogonal to said shaft of the expandable rod positioner.

15. A pedicle trocar with a sharp leading end for penetration, and a shaft and trailing end which are radiolucent.

16. The calibration device in claim 4, which specifies the length of the connecting rod to be utilized in the final construct.

17. The spinal stabilizing mechanism of claim 1 where the elongated connecting rod has leading and trailing ends which are engageable to cradles in trailing ends of bone fastening screws.

18. The spinal stabilizing mechanism of claim 17 where said leading and trailing ends are provided with a rotatable cam-shaft-disc mechanism.

19. The spinal stabilizing mechanism of claim 2 wherein said cradles are provided with apertures in the base of their central channels, these apertures specifically constructed so as to couple with the cam mechanism provided to the leading and trailing ends of the elongated connecting rod.
